SEQUENCE LISTING (11 sequences)

SEQ ID NO: 1 - protein, 5 aa, synthetic construct
GRGDS

SEQ ID NO: 2 - protein, 15 aa, synthetic construct
TRYLRIHPQSWVHQI

SEQ ID NO: 3 - protein, 12 aa, synthetic construct
HHLGGAKQAGDV

SEQ ID NO: 4 - protein, 13 aa, synthetic construct
RLMTQDCLQQRSK

SEQ ID NO: 5 - protein, 9 aa, synthetic construct
RTLAFVRFK

SEQ ID NO: 6 - protein, 4 aa, synthetic construct
RGDF

SEQ ID NO: 7 - protein, 21 aa, synthetic construct
GPOGPOGPOGPOGPOGPOGPO

SEQ ID NO: 8 - protein, 12 aa, synthetic construct
  VARIANT 2: note = Any aromatic Amino acid - W, Y, or F
  VARIANT 10: note = Any aromatic Amino acid - W, Y, or F
  VARIANT 7: note = X - G or D
XXXCPYXLCXIX

SEQ ID NO: 9 - protein, 11 aa, synthetic construct
XXCXYYGTCLX

SEQ ID NO: 10 - protein, 16 aa, synthetic construct
NHGCYNSYGVPYCDYS

SEQ ID NO: 11 - protein, 9 aa, synthetic construct
  DISULFID 1..9: note = cysteine cysteine disulfide cyclization
  MOD_RES 8: note = Ethoxy (OET) group
CNPRGDYRC

(19) United States
(12) Patent Application Publication
(10) Pub. No.: US 2023/0406905 A1
(43) Pub. Date: Dec. 21, 2023

(54) HEMOSTATIC NANOPARTICLES FOR THE TREATMENT OF NON-COMPRESSIBLE HEMORRHAGE AND INTERNAL BLEEDING

(71) Applicants: Massachusetts Institute of Technology, Cambridge, MA (US); The General Hospital Corporation, Boston, MA (US)

(72) Inventors: Paula T. Hammond Cunningham, Newton, MA (US); Bradley D. Olsen, Arlington, MA (US); George Velmahos, Boston, MA (US); Celestine Jia Huey Hong, Cambridge, MA (US)

(21) Appl. No.: 18/169,695

(22) Filed: Feb. 15, 2023

Related U.S. Application Data
(60) Provisional application No. 63/321,125, filed on Mar. 18, 2022.

(51) Int. Cl. C07K 14/745; A61K 47/10; A61P 7/04

(52) U.S. Cl. CPC C07K 14/745; A61K 47/10; A61P 7/04

(57) ABSTRACT

An injectable nanoparticular formulation and method of use thereof for treating non-compressible hemorrhage or internal bleeding has been developed. The formulation includes two interactive components, one a targeting nanoparticle with a polypeptide sequence that binds to a cell present at a site of injury, and the other a crosslinking nanoparticle with a bioorthogonal click-crosslinking group.

### CROSS REFERENCE TO RELATED APPLICATION

This application claims priority to U.S. Provisional Patent Application No. 63/321,125 filed Mar. 18, 2022, the disclosure of which is incorporated herein by reference.

### STATEMENT REGARDING FEDERALLY SPONSORED RESEARCH OR DEVELOPMENT

This invention was made with government support under W911NF-18-2-0048 and W81XWH-18-2-0010 awarded by the Army Research Office. The government has certain rights in the invention.

### FIELD OF THE INVENTION

This is generally in the field of hemostatic agents, and more specifically formulations of injectable crosslinkable nanoparticles targeted to activated platelets, and crosslinkers thereof, to effect hemostasis.

### REFERENCE TO THE SEQUENCE LISTING

The Sequence Listing submitted as a text file named “MIT_24141_US_ST26.xml” created on Jun. 26, 2023, and having a size of 12,633 bytes is hereby incorporated by reference pursuant to 37 C.F.R. § 1.834(c)(1).

### BACKGROUND OF THE INVENTION

Traumatic hemorrhage contributes to upwards of 2.5 million casualties per year, resulting in a significant number of deaths in both civilian and military populations. The process of achieving hemostasis in response to traumatic bleeding involves several steps: the activation of platelets via exposed collagen, the binding of activated platelets to von Willebrand factor, collagen, and fibrinogen to form a platelet plug (primary hemostasis), the intrinsic and extrinsic pathways of coagulation that culminate in thrombin generation, and the cleavage of fibrinogen via thrombin into self-polymerizable fibrin (secondary hemostasis). These steps are all mechanistically interconnected: for instance, fibrinogen-platelet binding aids in the formation of a platelet plug, while an activated platelet provides the surface on which prothrombin is converted to thrombin, which leads to fibrin crosslinking.

Several solutions in literature have been proposed to accelerate hemostasis through interactions with these wound-specific components. For topical wounds, these include materials such as positively-charged chitosan, which can cause nonspecific aggregation of platelets and red blood cells, or self-assembling peptide coatings such as RADA16 that mimic the structure of crosslinked fibrin. For internal wounds, these include intravenously-delivered linear polymers conjugated with fibrin-specific or von Willebrand-binding peptides^{[1]}, as well as polymer and liposomal nanoparticles functionalized with platelet-aggregating peptides. Though certain challenges remain, such as complement activation in response to nanoparticle infusion and nonspecific accumulation in lung and clearance organs, these materials have been demonstrated to significantly increase survival in a wide variety of animal models and present valuable options for the treatment of internal hemorrhage.

Hemostasis is achieved through a multitude of interactions involving both protein and cellular components of the wound microenvironment. However, the materials which have been proposed to promote hemostasis have either focused on enhancing the platelet-aggregating aspect of primary hemostasis, increasing the stability of the fibrin clot formed in secondary hemostasis, or recovering thrombin generation. This neglects the key components of the process when fibrinogen concentration, clot stability, platelet function, and platelet availability are adversely impacted during traumatic blood loss and subsequent fluid resuscitation.

For instance, while fibrin-crosslinking polymers may slow the degradation of clots via plasminolysis, platelets have also been demonstrated to be instrumental in overall clot strength. Therefore; while increased platelet number has been associated with higher rates of clot formation and thrombin generation^{[2]}, it fails to recover fibrinogen depleted through blood loss, even though fibrinogen deteriorates below critical levels ahead of other coagulation factors. Moreover, the lethal triad of trauma triggered by acute hemorrhage—hypothermia, coagulopathy, and acidosis—exacerbates fibrinogen degradation and decreases platelet activation^{[3]}, which may be difficult to counter solely by enhancing a discrete portion of hemostasis.

These points illustrate the multifarious effects of traumatic bleeding on the coagulation system, underscoring the need for a more comprehensive solution.

Primary hemostasis (formation of a platelet plug) and secondary hemostasis (formation of a fibrin clot) are two simultaneous, mechanistically intertwined processes that occur upon vascular injury. Researchers in the field have sought to treat internal hemorrhage by leveraging cues specific to these processes, such as using peptides that bind activated platelets or cleaved fibrinogen to target the wound site. While such materials have been demonstrated to significantly increase survival in a variety of injury models, they are commonly designed for the exclusive purpose of treating primary or secondary hemostasis.

It is therefore an object of the present invention to provide formulations for treatment of both primary and secondary hemostasis.

It is a further object of the present invention to provide formulations which can be injected to treat deep internal bleeding, where compression or immediate wound closure is not possible.

### SUMMARY OF THE INVENTION

Primary hemostasis (formation of a platelet plug) and secondary hemostasis (formation of a fibrin clot) are two simultaneous, mechanistically intertwined processes that occur upon vascular injury. An injectable formulation has been developed which includes a targeting component on nanoparticles and a crosslinking component. The targeting component binds the nanoparticles to activated platelets, and the cross-linking agent binds the nanoparticles to form the activated platelets into a hemostatic mass at a site of bleeding. The targeting ligand specifically binds activated platelets or other targets on the surface of endothelial cells present at areas of vascular injury such as von Willebrand Factor (vWF). This avoids formation of a cell aggregate enhancing clot formation anywhere but at the area of vascular injury, thereby increasing safety and efficacy. In the preferred embodiment, the targeting component is a ligand for a receptor on a cell present at a site of injury, most preferably a peptide which binds to a receptor on an activated platelet or von Willebrand factor, such as a peptide including the sequence GRGDS (SEQ ID NO:1). These ligands are typically peptides between four and twenty amino acids (200-3300 Da).

In the preferred embodiment, the targeting agent is bound to nanoparticles formed of polymers including a hydrophilic component and a hydrophobic component, wherein the peptide sequence is covalently attached to the hydrophilic component. A preferred hydrophilic component is a polyethylene glycol (PEG) molecule. The hydrophilic component is bound to the hydrophobic component forming the nanoparticles. In a preferred embodiment, the hydrophobic component is a polyester, most preferably poly(D,L-lactide-co-glycolide (PLGA). The nanoparticles are preferably between 50 and 400 nm, most preferably between 100 and 350 nm. The exemplified nanoparticles have an average diameter of 120 nm. Other agents that could be used include liposomes and inorganic particles such as metal nanoparticles.

The crosslinking component includes a moiety which reacts with the targeting component. In a preferred embodiment, the moiety reacting with the targeting component is a bioorthogonal click-crosslinking group such as an azide. In the most preferred embodiment, the bioorthogonal click-crosslinking group is a dibenzylcyclooctyne (DBCO).

In another preferred embodiment, the crosslinking component includes a first chemical moiety that reacts with a second chemical moiety on nanoparticles containing a component (e.g., a polypeptide sequence) that targets activated platelets or a von Willebrand factor. In a preferred embodiment, the first chemical moiety reacting with the second chemical moiety is a bioorthogonal crosslinking group (e.g., a bioorthogonal click-crosslinking group such as an azide or an alkyne). In the most preferred embodiment, the bioorthogonal click-crosslinking group is an alkyne-containing group, such as dibenzylcyclooctyne (DBCO). Further, the DBCO group is preferably conjugated to a hydrophilic polymer, such as PEG. The PEG can be a multi-arm PEG or PEG forming part of a nanoparticle.

The targeting component and the crosslinking agent are packaged separately in a kit for administration when needed. The two components are preferably administered sequentially, at the same site and time, or close thereto. They should not be mixed together prior to or at the time of administration. In the preferred embodiment, the components are administered intravenously, although they may be administered at a site using a catheter, or topically or into a lumen or wound. In one embodiment, the formulation is administered by injection or infusion to treat non-compressible hemorrhage. In one embodiment, the subject has internal bleeding. The composition localizes at the site of the internal injury. The composition may lower the threshold for platelet accumulation and/or increase clot stability, and/or decrease blood loss.

The examples demonstrate the synthesis of an interactive two-component system composed of (1) a targeting component such as a mixed azide-peptide targeting peptide such as GRGDS (SEQ ID NO:1) bound to PEG-b-PLGA nanoparticles and (2) a crosslinking component such as dibenzylcyclooctyne-functionalized multi-arm PEG or DBCO-PEG-b-PLGA nanoparticles. These were shown to be useful for the treatment of internal bleeding, both amplifying platelet recruitment and mitigating plasminolysis for greater clot stability. The functionality of the copper-free azide-DBCO click reaction was first validated by tracking nanoparticle aggregation via dynamic light scattering relative to unfunctionalized controls. Multiple azide: GRGDS mass ratios were also screened to determine the preferred combination most suitable for in vivo experiments, and a significant increase in both nanoparticle binding and platelet recruitment was observed relative to the nanoparticle-only system for an azide: GRGDS ratio of 1:3, i.e., GRGDS: azide ratio of 3:1. This ratio was used in biodistribution and biocompatibility studies, which confirmed that the non-targeted crosslinking particles did not aggregate in the lungs or significantly skew the accumulation of the targeted nanoparticles. The two-component system was challenged with a mouse liver resection model at a decreased nanoparticle dose of 0.5 mg/mL, and was shown to result in a significant (P=0.0099**) increase in survival relative to the particle-only control. Studies in a pig model also demonstrated safety and dosage scaleup.

### BRIEF DESCRIPTION OF THE DRAWINGS

FIG. 1A is a schematic of clot formation. FIGS. 1B-1F show synthesis schemes for each of GRGDS-PEG-b-PLGA (FIGS. 1B, 1C), N3-PEG-PLGA (FIG. 1D), and DBCO-PEG-PLGA (FIGS. 1E, 1F), nanoparticles (NP) through ring-opening polymerization and conjugation chemistry. FIG. 1G is a graph of size distribution of NPs, showing Intensity (%) over diameter (nm) (PDI<0.2).

FIGS. 2A-2J are graphs of nanoparticle (NP) and platelet recruitment over multiple rounds of incubation as determined via the LDH assay. FIGS. 2A-2C are graphs of normalized fluorescence reading showing GRGDS-azide nanoparticle (G) accumulation for each of nanoparticle only (All G), G/N Mass Ratio of 5:1 (G/N=5), G/N Mass Ratio of 3:1 (G/N=3), G/N Mass Ratio of 1:1 (G/N=1), (All N), and unfunctionalized, each with DBCO (full bar) or without DBCO (hashed bar), respectively, when using a polymer crosslinker (N) in the described concentrations at 2 incubations (FIG. 2A), 4 incubations (FIG. 2B), or 6 incubations (FIG. 2C), respectively. FIGS. 2D-2F are graphs of Normalized fluorescence reading showing platelet accumulation for each of GRGDS-azide nanoparticle (G) only (All G), G/N Mass Ratio of 5:1 (G/N=5), G/N Mass Ratio of 3:1 (G/N=3), G/N Mass Ratio of 1:1 (G/N=1), (All N), and unfunctionalized, each with DBCO (full bar) or without DBCO (hashed bar), respectively, when using a polymer crosslinker (N) in the described concentrations at 2 incubations (FIG. 2D), 4 incubations (FIG. 2E), or 6 incubations (FIG. 2F), respectively. FIGS. 2G-2H are graphs of Normalized fluorescence reading showing nanoparticle accumulation for each of GRGDS-azide nanoparticle (G) only (All G), G/N Mass Ratio of 5:1 (G/N=5), G/N Mass Ratio of 3:1 (G/N=3), G/N Mass Ratio of 1:1 (G/N=1), (All N) and unfunctionalized, each with DBCO (full bar) or without DBCO (hashed bar), respectively, when using a nanoparticle crosslinker (N) in the described concentrations at 2 incubations (FIG. 2G), and 4 incubations (FIG. 2H), respectively. G/N=3P is also shown. G/N=3 in this particular experiment refers to a nanoparticle crosslinker, and G/N=3P was labeled as such to emphasize that this is the polymer crosslinker. FIGS. 2I-2J are graphs of normalized fluorescence reading showing platelet accumulation for each of GRGDS-azide nanoparticle (G) only (All G), G/N Ratio of 5:1 (G/N=5), G/N Ratio of 3:1 (G/N=3), G/N Ratio of 1:1 (G/N=1), (All N), and unfunctionalized, each with DBCO (full bar) or without DBCO (hashed bar), respectively, when using a nanoparticle crosslinker (N) in the described concentrations at 2 incubations (FIG. 2I), and 4 incubations (FIG. 2J), respectively. G/N=3P is also shown, to show addition of a cross-linkable component increases NP and platelet accumulation over multiple incubations. The original NP system is marked (boxed). For all tests, ns: p>0.05; *: p≤**: p≤0.01; ***: p≤0.001 as determined through 2-way ANOVA with Bonferroni post-tests.

FIGS. 3A-3H are graphs of particle size graphed for different two-component systems composed of combinations of pure azide nanoparticles (NPP) and Azide-GRGDS nanoparticles (GNNP), showing Diameter (nm) over time (h) as confirmed through NP aggregation in dynamic light scattering (DLS) measurements for concentrations of 50 mg (•); 20 mg (▪); 10 mg (▴); 5 mg (▾); and 2 mg ( [symbol] ), respectively, for each of N3-PEG-PLGA (NPP) with DBCO-PEG-PLGA (DPP) (FIG. 3A); NPP with Methoxy-PEG-PLGA (MPP) (FIG. 3B); NPP with 4-Arm-DBCO-PEG (4ADP) (FIG. 3C); NPP with 4-Arm-PEG (4ADP) (FIG. 3D); Mixed GRGDS-N₃-PEG-PLGA (GNPP) with DPP (DPP) (FIG. 3E); GNPP with GDPP (Mixed GRGDS-DBCO-PEG-PLGA) (FIG. 3F); GNPP with 4ADP (FIG. 3G); and GNPP with MPP (FIG. 3H). FIG. 3I is a graph of particle stability over 72 hours in deionized water, Diameter (nm) over time in days, for each of GRGDS-azide NPs (GNPP; •), GRGDS-NPs (GPP; ▪) and DBCO-NPs (DPP; ▴), respectively. FIG. 3J is a graph of particle stability with stoichiometric crosslinking of the two-components, showing diameter (nm) over time in hours/days, for different azide-to-DBCO (N/D) ratios, including of N/D=1 (•), N/D=0.75 (▪) and N/D=1.25 (▴), respectively.

FIGS. 4A-4D depict decreasing doses of two-component system resulting in increased specific platelet accumulation relative to a nanoparticle-only system; addition of a cross-linkable component lowers threshold for platelet accumulation and increases clot stability. FIG. 4A is a schematic showing serial dilutions of nanoparticles (NP) into platelet rich plasmin (PRP). FIG. 4B is a histogram of changes in complement levels, showing C5a Fold-change relative to glucose control for each of GRGDS-azide NPs at 1 mg/ml, GRGDS-azide NPs at 0.5 mg/ml, two-component system using polymeric cross-linker (Two-component (P), two-component system using nanoparticle cross-linker (Two-component (NP)), and zymosan, respectively. FIGS. 4C and 4D are histograms of Normalized Absorbance readings for each of Pure GRGDS NPs, Two-component (P) and Two-component (NP) at each of 1 mg/ml, 0.5 mg/ml and 0.25 mg/ml, respectively, at each of 2 incubations (FIG. 4C), and 4 incubations (FIG. 4D), respectively. For C5a levels, n=5, ns: p>0.05; *: p≤0.05; **: p≤0.01; ***: p≤0.001 as determined through one-way ANOVA with Tukey's post-test for trial groups (without Zymosan); for all other tests, n=6, ns: p>0.05; *: p≤0.05; **: p≤0.01; ***: p≤0.001 as determined through two-way ANOVA with Tukey's post-test.

FIGS. 5A-5C depict the two-component system recovering platelet recruitment in hemodiluted conditions. FIG. 5A is a schematic of the procedure showing serial dilutions of PRP by 20%. FIGS. 5B and 5C are histograms of Normalized Absorbance Values over Plasma dilution for each of Pure GRGDS NPs (light, full bar), Two-component (P) (hashed bar) and Two-component (NP) (dark, full bar), respectively, showing the two-component system is capable of recovering platelet recruitment in diluted PRP relative to the single component system at higher plasma concentrations for two incubations (FIG. 5B) and four incubations (FIG. 5C), respectively. FIGS. 5D-5E are histograms of nonspecific platelet binding showing Normalized Absorbance Values over various nanoparticle concentrations (1 mg/ml; 0.5 mg/ml; and 0.25 mg/ml, respectively) for each of Pure GRGDS NPs (light, full bar), Two-component (P) (hashed bar) and Two-component (NP) (dark, full bar), respectively, at each of two incubations (FIG. 5D) and at four incubations (FIG. 5E), respectively. FIGS. 5F-5G are histograms of nonspecific platelet binding showing Normalized Absorbance Values over various plasma dilutions (0%, 20%, 40%, respectively) for each of Pure GRGDS NPs (light, full bar), Two-component (P) (hashed bar) and Two-component (NP) (dark, full bar), respectively, at each of two incubations (FIG. 5F) and at four incubations (FIG. 5G), respectively. For all tests, n=6, ns: p>0.05; *: p≤0.05; **: p≤0.01; ***: p≤0.001 as determined through two-way ANOVA with Tukey's post-test.

FIGS. 6A-6F are graphs showing the two-component system increases fibrin crosslinking and decreases clot plasminolysis by ˜40% in dilutional coagulopathic clotting conditions. FIG. 6A is a graph of representative absorbance over time for each of control (•), GRGDPS (∇), Two-component (P) (♦) and Two-component (NP) (▪), respectively. FIG. 6B is a histogram of overall coagulation potential for each of control, Two-component (P), Two-component (NP), and GRGDPS, respectively. The overall coagulation potential is significantly increased for the two-component system. FIG. 6C shows normalized transmission (%) over time for each of control (•), GRGDPS (∇), Two-component (P) (♦) and Two-component (NP) (▪), respectively, depicting the amount of fibrinogen coagulation for each. FIG. 6D is a histogram of change in Transmission (%) for each of control, Two-component (P), Two-component (NP), and GRGDPS, respectively; crosslinking is significantly increased with the two-component system relative to the single-component system and the control. FIG. 6E is an image showing fibrin crosslinking in different conditions, showing Lightening and movement of interface in control clot (top: saline; bottom: plasmin). FIG. 6F is a graph of degradation profiles of fibrin clot, nanoparticle+fibrin clot, and two-component system+fibrin clot, showing normalized fluorescent intensity over time (hours) for each of fibrin clot (•) GRGDS-NP (▪), and Two-component (NP) (▴), respectively. For (a)-(d), n=12, ns: p>0.05; *: p≤0.05; **: p≤0.01; ***: p≤0.001 as determined through one-way ANOVA with Tukey's post-test; for (e)-(f), n=5, ns: p>0.05; *: p≤0.05; **: p≤0.01; ***: p≤0.001 as determined through two-way ANOVA with Tukey's post-test.

FIGS. 7A-7F are graphs showing biodistribution and pharmacokinetics of the two-component system at various crosslinker concentrations relative to nanoparticle-only groups. FIG. 7A is a histogram of retention of the two-component system and controls, showing Normalized fluorescence readings for each of No DBCO; 5 mg/ml; 10 mg/ml; 15 mg/ml; 20 mg/ml; 1:1 DBCO NP and Pure GRGDS, as indicated, at each of times 0, 40, 80, 120, and 150 minutes. FIG. 7B is a graph of blood circulation concentration of the two-component system and controls, showing NP concentration in blood (%) for each of No DBCO; DBCO at 5 mg/ml; 10 mg/ml; 15 mg/ml; and 20 mg/ml; 1:1 DBCO NP and Pure GRGDS, respectively, as indicated. FIGS. 7C-7D show biodistribution that is not normalized to mass depicted by a histogram of Normalized total radiant efficiency (FIG. 7C), and biodistribution normalized to mass depicted by a histogram of Normalized total radiant efficiency per unit mass (FIG. 7D) for each of GNPP, GNPP+UF, GNPP+DBCO10, GNPP+DBCO20 and GNPP+DPP in each of the organs kidney, lung, heart, spleen and liver. FIGS. 7E and 7F are histograms showing inflammatory cytokine levels for each of blank, GPP, GNPP+DPP, GNPP, GNPP+ 5 mg/ml 4ADP, GNPP+ 10 mg/ml 4ADP, GNPP+ 15 mg/ml 4ADP and GNPP+ 20 mg/ml 4ADP, for each of interleukin-6 (IL-6) (FIG. 7E) and TNF-alpha (FIG. 7F), respectively.

FIGS. 8A-8E depicts hemostatic efficacy of the two-component system in a lethal mouse liver resection model. FIG. 8A is a schematic of the procedure; FIG. 8B is a graph of accumulation in resected vs remnant liver, showing normalized fluorescence per mass for each of resected (open bar) vs remnant liver (full bar) for each of GRGDPS-NP 0.5 mg/ml, Two-component (P) and, Two-component (NP), respectively. FIG. 8C is a graph of blood loss, showing fluid loss (%) for each of Two-component (P) and, Two-component (NP), GRGDPS-NP 1 mg/ml, and GRGDPS-NP 0.5 mg/ml, respectively. FIG. 8D is a graph of survival over time (0-3 hours) for each of Two-component (P) and, Two-component (NP), GRGDPS-NP 1 mg/ml, and GRGDPS-NP 0.5 mg/ml, respectively. ns: p>0.05; *: p≤0.05; **: p≤0.01; ***: p≤0.001 as determined through Kaplan-Meier analysis and two-way ANOVA. FIG. 8E is a histogram of organ biodistributions for GRGDS-NP at 0.5 mg/mL vs. two-component system at 0.5 mg/mL GNPP with polymers (P) and nanoparticle crosslinkers (NP), showing normalized fluorescence per mass for each of resected liver, remnant liver, lungs, kidneys, spleen, heart, and blood/clots for Two-component (P) and, Two-component (NP), GRGDPS-NP (0.5 mg/ml).

FIGS. 9A-9D are graphs depicting heart rate (HR) and mean arterial pressure (MAP), following crosslinker and NP injection, showing each of heart rate and mean arterial pressure (HR/MAP) as a function of time following injection of GRGDS-NP at 1 mg/kg (FIG. 9A); GCV-NP (Nanoparticles functionalized with a mixture of GRGDS, cyclic GRGDS, and vWF-binding peptide (VBP)) at 1 mg/kg (FIG. 9B); GRGDS-NP at 2 mg/kg (FIG. 9C) and GCV-NP at 2 mg/kg (FIG. 9D). No fluctuations were observed, though transient fluctuations were observed in the few minutes (at t=10 min) following crosslinker injection (t=5 min).

FIGS. 10A-10D are graphs depicting oxygen saturation (spO2) and end tidal carbon dioxide (EtCO2), following crosslinker and NP injection, showing each of oxygen saturation (spO2) and end tidal carbon dioxide (EtCO2) as a function of time following injection of GRGDS-NP at 1 mg/kg (FIG. 10A); GCV-NP at 1 mg/kg (FIG. 10B); GRGDS-NP at 2 mg/kg (FIG. 10C) and GCV-NP at 2 mg/kg (FIG. 10D). No changes in oxygen saturation and end-tidal carbon dioxide was observed following NP injection (t=10 min) or crosslinker injection (t=5 min).

### DETAILED DESCRIPTION OF THE INVENTION

### I. Definitions

The term “primary hemostasis” refers to the formation of a platelet plug. When an injury occurs, collagen and von Willebrand Factor (vWF) is exposed on the subendothelial matrix, allowing activated platelets to bind. Activated platelets can also bind fibrinogen, which binds multiple platelets at once, leading to platelet aggregation and the formation of the platelet plug.

The term “secondary hemostasis” relates to the formation of a fibrin clot generated through the coagulation cascade. The intrinsic and extrinsic pathways culminate in the generation of thrombin, which cleaves circulating fibrinogen to form fibrin. Fibrin is able to self-polymerize to form a gel.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

Nanoparticle (“NP”), as used herein, generally refers to a particle of any shape having a diameter from about 1 nm up to, but not including, about 1 micron, more typically from about 5 nm to about 500 nm. NPs having a spherical shape are generally referred to as “nanospheres”.

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the material degrades or breaks down into its component subunits, or digestion, e.g., by a biochemical process, of the material into smaller (e.g., non-polymeric) subunits.

Many pharmaceutically acceptable biocompatible polymers are available, including polyhydroxyesters such as polylactic acid, polyglycolic acid, and copolymers of lactic acid and glycolic acid, polyanhydrides, polyorthoesters, polyhydroxybutyrates, as well as non-biodegradable polymers such as acrypolyacrylates, polyurethane, and. Although proteins and polysaccharides can be used, these are not preferred.

Copolymers of polyalkylene oxide (polyethlene glycol or PEG) or derivatives thereof with any of the polymers described above may be used to make the polymeric particles. In certain embodiments, the PEG or derivatives may be located in the interior positions of the copolymer. Alternatively, the PEG or derivatives may locate near or at the terminal positions of the copolymer. For example, one or more of the polymers above can be terminated with a block of polyethylene glycol. In some embodiments, the core polymer is a blend of pegylated polymer and non-pegylated polymer, wherein the base polymer is the same (e.g., PLGA and PLGA-PEG) or different (e.g., PLGA-PEG and PLA). In certain embodiments, the NPs are formed under conditions that allow regions of PEG to phase separate or otherwise locate to the surface of the particles. The surface-localized PEG regions alone may perform the function of, or include the surface-altering agent. In particular embodiments, the particles are prepared from one or more polymers terminated with blocks of polyethylene glycol as the surface-altering material.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to Daltons, 5,000 Daltons to 50,000 Daltons, or 5,000 Daltons to Daltons.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

As used herein, bioorthogonal reactions enable the study of biomolecules such as glycans, proteins, and lipids in real time in living systems without cellular toxicity. A number of chemical ligation strategies have been developed that fulfill the requirements of bioorthogonality, including the 1,3-dipolar cycloaddition between azides and cyclooctynes (also termed copper-free click chemistry), between nitrones and cyclooctynes, oxime/hydrazone formation from aldehydes and ketones, the tetrazine ligation, the isocyanide-based click reaction, and most recently, the quadricyclane ligation. The use of bioorthogonal chemistry typically proceeds in two steps. First, a cellular substrate is modified with a bioorthogonal functional group (chemical reporter) and introduced to the cell; substrates include metabolites, enzyme inhibitors, etc. The chemical reporter must not alter the structure of the substrate dramatically to avoid affecting its bioactivity. Secondly, a probe containing the complementary functional group is introduced to react and label the substrate.

“Covalent linkage” refers to a bond or organic moiety that covalently links particles, macromolecules, or both, in a layer adjacent to the surface of a cell to a molecule on the cell's surface. “Covalent linkage” can also refer to a bond or organic moiety that covalently links particles, macromolecules, or both, in one layer to particles, macromolecules, or both, in an adjacent layer in the conformal coating on the surface of a cell.

The term “analog” refers to a chemical compound with a structure similar to that of another “reference” compound, but differing from it in respect to a particular component, functional group, atom, etc.

The term “derivative” refers to a compound, which is formed from a parent compound by one or more chemical reaction(s).

Surfactant is a general name for substances that absorb to surfaces or interfaces to reduce surface or interfacial tension. These agents aid wetting and dispersion of hydrophobic active pharmaceutical ingredients and they usually act by reducing the interfacial tension between solids and liquids in suspensions.

“Excipient” is used herein to include a pharmaceutically acceptable compound that is not a therapeutically or biologically active compound. An excipient should generally be inert and non-toxic to the subject.

The terms “biocompatible” and “biologically compatible” generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The terms “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated a disease or disorder of the eye is mitigated or eliminated.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control.

### II. Formulations

There are two components: wound targeting and crosslinking.

A. Wound Targeting Particles

Nanoparticles

The nanoparticles are formed of biocompatible polymers that include a hydrophilic component such as (poly(ethylene glycol)) and a hydrophobic component such as biocompatible synthetic polymers such as polyesters, polyacrylates, and polyurethanes.

Synthesis: These nanoparticles can be synthesized from commercial starting materials, though the method through which they can be synthesized will differ with the choice of starting materials. Custom peptide sequences and monomers and macroinitiators used for polymerization can be used to produce the materials through techniques such as ring-opening polymerization, controlled free radical polymerization, interfacial polymerization, and conjugation chemistries.

Nanoparticles can also be made from solid materials such as metals (such as gold, iron) or inert inorganic materials by milling, fracturing, or other standard techniques.

Liposomes, Micelles, Nanoemulsions and Lipid Nanoparticles

Liposomes are a supramolecular aggregate made from amphipathic molecules, typically phospholipids, formed in an aqueous phase. They can range from tens of nanometers to hundreds of micrometers in size. Due to their size and hydrophobic and hydrophilic character and biocompatibility, liposomes are commonly used for drug delivery. Liposome properties differ considerably with lipid composition, surface charge, size, and the method of preparation, so they can be modified as needed for different applications. The choice of bilayer components determines the ‘rigidity’ or ‘fluidity’ and the charge of the bilayer. For instance, unsaturated phosphatidylcholine species from natural sources (egg or soybean phosphatidylcholine) give much more permeable and less stable bilayers, whereas the saturated phospholipids with long acyl chains (for example, dipalmitoylphosphatidylcholine) form a rigid, rather impermeable bilayer structure.

There are a number of techniques to make liposomes, and variations thereof. Single emulsions use surfactants to suspend drops of oil in water or drops of water in oil to form liposomes. Micelles are particles made up of surfactants that have cores composed of lipophilic tails. Nanoemulsions are particles that have an oil core; surrounded by a layer of surfactant. Solid lipid nanoparticles have a solid wax or lipid core surrounded by a layer of surfactants. Nanostructured lipid carriers contain a blend of solid and liquid lipids at the core.

All the methods of preparing the liposomes involve four basic stages:

Drying down lipids from organic solvent; dispersing the lipid in aqueous media; purifying the resultant liposome; and analyzing the final product.

B. Targeting Components

The targeting components have two parts. The first is a nanoparticle which is subsequently crosslinked with other targeted nanoparticles at the site of injury or bleeding. The second is a peptide bound to the nanoparticles which selectively target activated cells and proteins at the site of injury or bleeding.

The nanoparticles are functionalized with polyvalent short peptide sequences that interact specifically with cells and proteins present at the injury site, such as activated platelets and von Willebrand factor exposed on injured endothelial cell surfaces, but not with uninjured cells or quiescent platelets, as well as a click-cross-linkable moiety that will enable them to react with a second clot-strengthening component. Agents that bind to secondary coagulation mechanisms such as fibrin are not preferred since these may be found distant from the site of injury.

In a preferred embodiment, the targeting agent is a peptide, typically between four and twenty amino acids in length (mw 200-3300 Da) such as GRGDS (SEQ ID NO:1), which binds to a receptor on activated platelets but not quiescent platelets. The peptide is chemically coupled to the polymer forming the nanoparticle, or to a lipid, using standard techniques. For example, GRGDS-PEG-b-PLGA is synthesized through ring-opening polymerization in a 4:1 dichloromethane (DCM): dimethylformamide (DMF) solution for 80 minutes and purified via precipitation and dialysis as described in Hong, 2022, ACS Nano.

Representative targeting peptides include:
Name: Von Willebrand Factor (VWF)-binding peptide (VBP)
Sequence: TRYLRIHPQSWVHQI (SEQ ID NO:2)
Derived from: Factor VIII, binds VWF's D′—D3 domain.

Mechanism of action (targeting): free VWF exists in plasma, but also binds activated platelets and collagen. The type of VWF that is released at injury sites is ultra-large VWF (the largest multimeric species) and is not usually observed in the blood of normal individuals. This type of VWF is stored in endothelial cells, more specifically in Weibel-Palade bodies.

References:

### VWF Background (Structure, Storage, Release, Etc.):

http://onlinelibrary.wiley.com/doi/10.1111/j.1365-2516.2008.01848.x/epdf

### VWF-Binding Peptides on Liposomes:

http://pubs.acs.org/doi/pdf/10.1021/bc300086d
http://pubs.acs.org/doi/pdf/10.1021/bm300192t
https://www.ncbi.nlm.nih.gov/pmc/articles/PMC4300948/
Name: H12

### Sequence: HHLGGAKQAGDV (SEQ ID NO:3)

Derived from: Fibrinogen
Mechanism of action (targeting): Recognizes specifically the active form of GPIIb/IIIa on activated platelets—this is the same site that RGD-based peptides target.

### References:

H12-Functionalized Particles (Liposomes and Microparticles):
http://onlinelibrary.wiley.com/doi/10.1111/j.1537-2995.2005.00173.x/epdf
http://www.sciencedirect.com/science/article/pii/S0378517311000457?via %3Dihub

### Name: RLM

### Sequence: RLMTQDCLQQRSK (SEQ ID NO:4)

Derived from: Factor VII (from Factor VII's binding site to TF)
Mechanism of action (targeting): Tissue factor (TF) is expressed by cells within the vessel wall; exposure of TF by breaking the endothelial barrier triggers the extrinsic pathway of coagulation. Low levels of TF may exist in the blood.

References:

Background on TF:
https://www.ncbi.nlm.nih.gov/pmc/articles/PMC2838713/
https://www.thieme-connect.com/DOI/DOI?10.1055/s-2006-933336
TF-binding peptides (used in nanofibers):
http://pubs.acs.org/doi/pdf/10.1021/acsnano.5b06025

### Name: RTL

### Sequence: RTLAFVRFK (SEQ ID NO:5)

Derived from: Factor VII
Mechanism of action (targeting): Same as above
References: Same as above.
Name: RGD/Fibrinogen-mimetic peptide (FMP)
Sequence: GRGDS (SEQ ID NO:1), cyclo-CNPRGDY(OEt)RC (cyclic RGD) (SEQ ID NO:11), RGDF (SEQ ID NO:6)
Derived from: Fibrinogen
Mechanism of action (targeting): Recognizes specifically the active form of GPIIb/IIIa on activated platelets, this is the same complex that H12 targets.

### References:

RGD-functionalized particles (liposomes and nanoparticles):
http://pubs.acs.org/doi/pdf/10.1021/bm300192t
http://stm.sciencemag.org/content/1/11/11ra22/tab-pdf
https://www.ncbi.nlm.nih.gov/pmc/articles/PMC3496064/http://pubs.acs.org/doi/pdf/10.1021/acsbiomaterials.5b00493
https://pubs.acs.org/doi/10.1021/acs.bioconjchem.5b00070

### Name: Collagen-Binding Peptide (CBP)

### Sequence: (GPO)7 (SEQ ID NO:7)

Derived from: Synthetic
Mechanism of action (targeting): Promotes adhesion to fibrillar collagen (collagen exposed when vessel wall is wounded).

### References:

http://pubs.acs.org/doi/pdf/10.1021/bc300086d
http://pubs.acs.org/doi/pdf/10.1021/bm300192t
https://www.science.org/doi/10.1126/sciadv.aba0588?url_ver=Z39.88-2003&rfr_id=ori:rid:crossreforg&rfr_dat=cr_pub %20%200pubmed

### Name: Fibrin-Binding Peptide (1-BP)

Sequence: Cyclic peptides XArXCPY(G/D)LCArIX (Ar=aromatic) (SEQ ID NO:8), X2CXYYGTCLX (SEQ ID NO:9), and
NHGCYNSYGVPYCDYS (SEQ ID NO:10), cyclized by disulfide bond between cysteines.
Derived from: Synthetic
Mechanism of action (targeting): Targets fibrin clot.

### References:

https://www.ncbi.nlm.nih.gov/pmc/articles/PMC3310290/
https://www.science.org/doi/full/10.1126/scitranslmed.3010383

C. Crosslinking Agents

The crosslinking agents are typically in the form of a multivalent polymer; bivalent polymer; multivalent small molecule, or bivalent small molecule. These react with the corresponding moiety on the nanoparticles of the targeting component to bind together the targeted activated platelets and cells.

“Multivalent” and “bivalent” refers to organic compounds that contain three or more reactive moieties and two reactive moieties, respectively. The reactive moieties can all be the same (homofunctional) or at least two are different (heterofunctional). Preferably, the crosslinking agent is in the form of a multivalent polymer (preferably homofunctional) or nanoparticle that has the corresponding moiety to react with the moities on the particles in the targeting component.

Preferably, the reactive moieties of the cross-linking agent are bioorthogonal crosslinking groups. Examples of bioorthogonal crosslinking groups include azides; alkynes (such as dibenzyl cyclooctyne, aza-dibenzyl cyclooctyne, bicyclononynes); alkenes (such as trans-cyclooctenes, norbornenes, vinylboronic acids, and alkylcyclopropens); tetrazines; nitrones; oximes; triarylphosphines; aminooxys; carbonyls; hydrazides; sulfonyl chlorides; maleimides; aziridines; nitriles; acryloyls; acrylamides; sulfones; vinyl sulfones; cyanates; thiocyanates; isocyanates; isothiocyanates; alkoxysilanes; dialkyl dialkoxysilanes; diaryl dialkoxysilanes; trialkyl monoalkoxysilanes; vinyl silanes; acetohydrazides; acyl halides; epoxides (such as glycidyls); carbodiimides; phosphoramidates; vinyl ethers; substituted hydrazines; alkylene glycol bis(diester)s, e.g. ethylene glycol bis(succinate); thioesters, e.g., alkyl thioester, α-thiophenylester, allyl thioester (such as allyl thioacetae, allyl thioproprionate); allyl esters (such as allyl acetate, allyl propionate); aryl acetates (such as phenacyl ester); orthoesters; sulfonamides, such as 2-N-acyl nitrobenzenesulfonamide; vinyl sulfides; or a combination thereof. More preferably, the reactive moieties are bioorthogonal click-crosslinking groups. Examples of bioorthogonal click-crosslinking groups are azides and alkynes, preferably strained alkynes such as dibenzylcyclooctyne (DBCO). The strained alkynes facilitate click-crosslinking in the absence of a metal catalyst, e.g., copper-free or ruthenium-free click chemistry. This crosslinking component will only react with targeted nanoparticles with the corresponding reactive group and not with other biological tissues. The “dibenzyl cyclooctyne” is referred to interchangeably as “dibenzocyclooctyne).

Other crosslinking agents can include groups capable of engaging in selective affinity protein interactions, such as biotin and streptavidin; biotin and avidin; and heterodimeric coiled coils, which are peptide pairs that will crosslink with each other.

D. Other Components

Other components that may be included in this system alongside or within the nanoparticles include the use of procoagulant therapeutics and imaging or tracking agents. Examples of procoagulant therapeutics include: tranexamic acid, aprotinin, textilin, chitosan, self-assembling peptides (RADA16, RATEA16, AC5, etc.), silica or clay nanoplatelets/Laponite, polyphosphates, fibrinogen, cryoprecipitate, coagulation factors, polyelectrolytes or other charged nanoparticles comprising of these polymers (gelatin, chitosan, hyaluronic acid etc.). Other procoagulant therapeutics could include: aprotinin, textilin, polyphosphates, thrombin, chitosan, and self-assembling peptides (RADA, etc.). See https://pubs.acs.org/doi/full/10.1021/acs.jmedchem.9b01060,
https://www.ncbi.nlm.nih.gov/pmc/articles/PMC8216384/,
https://pubs.acs.org/doi/10.1021/nn503719n,
https://pubs.acs.org/doi/10.1021/acsnano.5b02374,
https://www.ncbi.nlm.nih.gov/pmc/articles/PMC6918334/, and
https://pubs.acs.org/doi/10.1021/acs.biomac.8b00588.

Examples of imaging agents include nano/microbubbles for ultrasound imaging, gold for computed tomography (CT), magnetic resonance imaging (MRI) contrast agents, radiocontrast agents, immunolabeling agents and near infra-red fluorescent agents. Other references for imaging agents:
https://www.ncbi.nlm.nih.gov/pmc/articles/PMC3032015/,
https://www.ncbi.nlm.nih.gov/pmc/articles/PMC6778549/,
https://pubmed.ncbi.nlm.nih.gov/33405762/,
https://www.ncbi.nlm.nih.gov/pmc/articles/PMC7602142/,
https://www.ncbi.nlm.nih.gov/pmc/articles/PMC3905628/, and
https://wires.onlinelibrary.wiley.com/doi/10.1002/wnan.1642.

Excipients for resuspending lyophilized or dried reagent include sterile diluents such as distilled water, saline, Lactated Ringer's, glucose/dextrose solutions, and colloids (ex: starches, dextrans, albumins, gelatin, etc.). Stabilizing agents: polymeric stabilizer (Pluronics/poloxamers, Polyvinylpyrrolidone (PVP) polysaccharides (gellan, pectin, k-carrageenan, etc.)), lyoprotectants (sucrose, trehalose, mannitol, maltose, lactose, fructose, etc.). See
https://www.sciencedirect.com/science/article/pii/S0168365916300311 and
https://www.sciencedirect.com/science/article/pii/S0168365916300311.

### III. Methods of Making

Polyethylene glycol (PEG)-b-poly(lactic-co-glycolic) (PLGA) nanoparticles were functionalized with wound-targeting peptides and azide bioorthogonal click groups. A second component, functionalized with the corresponding copper-free click moiety dibenzylcyclooctyne (DBCO), was delivered with the aim of crosslinking the targeted nanoparticles through a fibrin-independent mechanism to achieve wound-targeted clot stabilization. This process is illustrated below in Scheme 1. Two versions of this second component were tested—a multiarm PEG-DBCO crosslinker, or a DBCO-functionalized nanoparticle crosslinker.

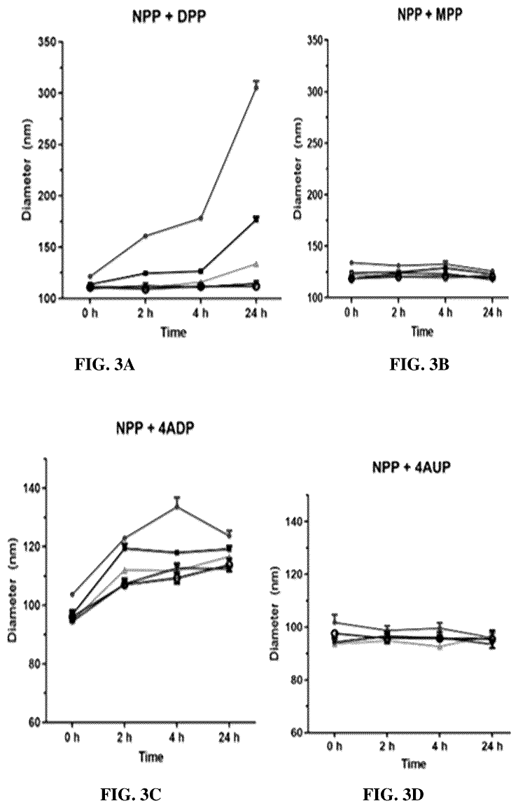

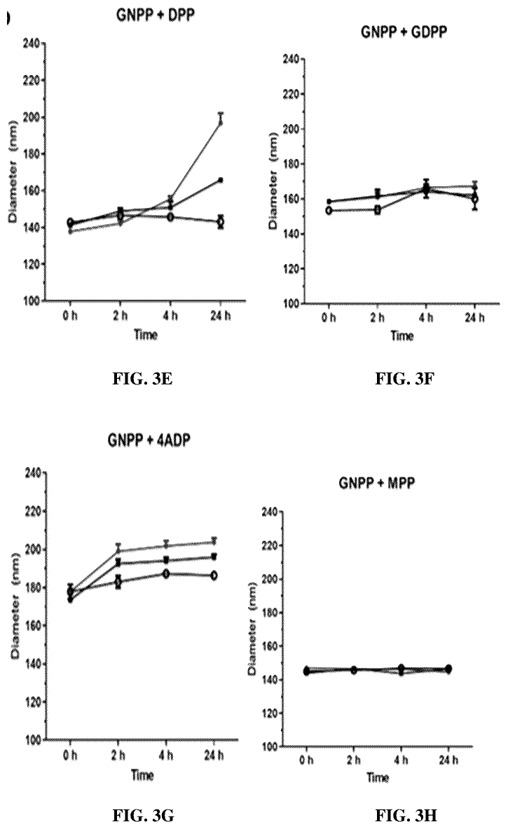

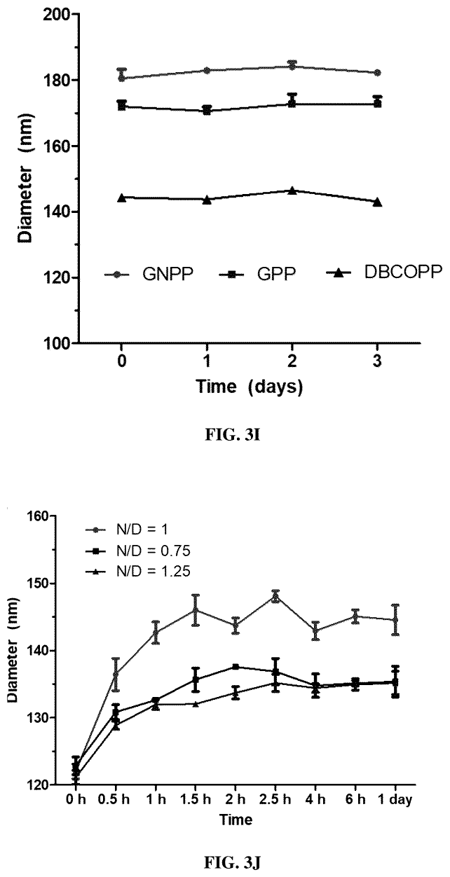

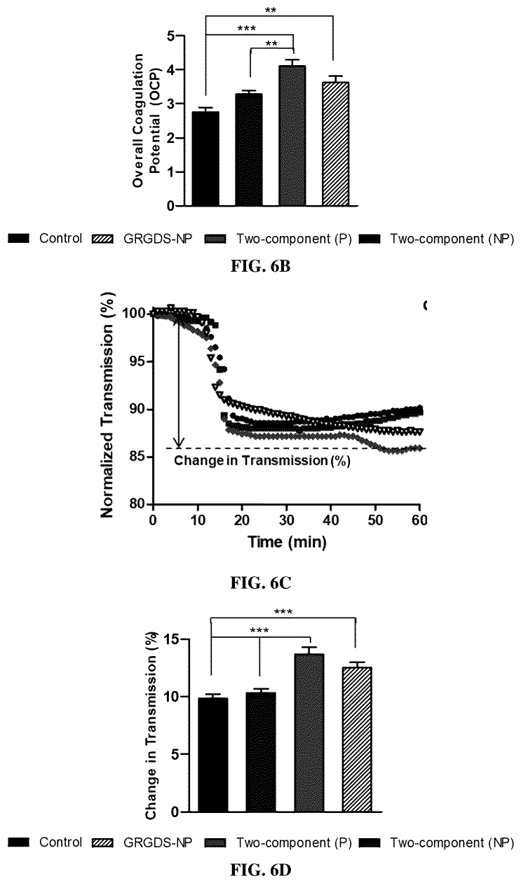

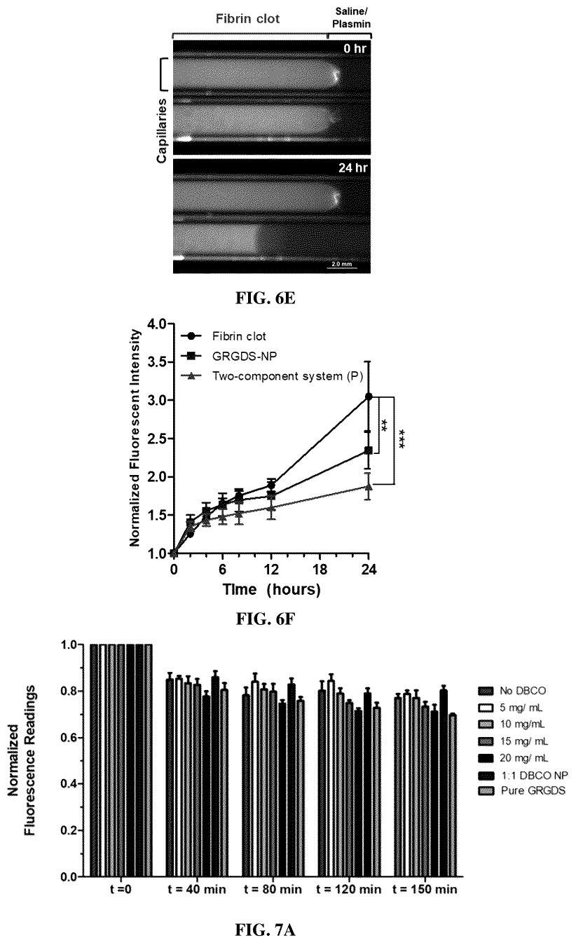

The results are shown in FIG. 6A-6H. As wound-targeting nanoparticles were observed in several studies to result in significantly higher accumulation at the wound site the aim of the two-component system was to result in significant crosslinking only at higher concentrations. The two-component system with either nanoparticle or multiarm polymer crosslinker was found to result in nanoparticle aggregation at higher concentrations but not at circulating concentration. Using dynamic light scattering to monitor crosslinking behavior, the functionality of the clickable groups was first evaluated using pure azide nanoparticles (FIG. 3a), before mixed nanoparticles were also tested at these concentrations (FIG. 3b). Representative inversion tests of the nanoparticle solutions at 200 mg/mL have also been provided in FIG. S3. Control groups of unfunctionalized nanoparticle (MPP) and unfunctionalized four-arm-PEG (4AUP) were included to confirm that any observed aggregation did not occur due to high nanoparticle concentration and subsequent sedimentation.

Pure azide-functionalized nanoparticles (FIG. 3A) demonstrated visible increases in size within the first two hours of the experiment when incubated with both nanoparticle and multiarm polymer crosslinkers at 50 mg/mL. No significant increases in sizes were observed at 5-10 times circulating concentration (1-0.5 mg/mL) for the nanoparticle+nanoparticle crosslinker combination (NPP+DPP), while a slight increase in size was observed in the nanoparticle+multiarm polymer system (NPP+4ADP). No size increases were observed with the control groups of unfunctionalized nanoparticle and unfunctionalized multiarm polymer. Mixed nanoparticles (GNPP) were also observed to increase in size at higher concentrations, though the kinetics appeared to be delayed in comparison to the pure azide nanoparticles, likely due to the lower percentage of crosslinkable functionalities (FIG. 3b). Two types of DBCO-functionalized nanoparticle crosslinkers were tested in this experiment—one of pure DBCO-PEG-b-PLGA (DPP), and one mixed with GRGDS-PEG-b-PLGA (GDPP). Nanoparticle aggregation behavior was observed to be much more pronounced in the GNPP+DPP solution despite the same amount of DBCO-functionalized polymer, a phenomenon that could be potentially attributed to the higher functionality per molecule of pure DBCO-functionalized nanoparticles. As a result, all further experiments were conducted with DPP instead of GDPP, to ensure crosslinking could still occur upon accumulation at the injury site. Only minimal increases in nanoparticle size were observed for the GNPP+4ADP combination at circulating concentration, and no increases in size were observed with unfunctionalized nanoparticles. Overall, the two-component system with either nanoparticle or multiarm polymer crosslinker led to significant nanoparticle aggregation at higher concentrations but not at circulating concentration.

### Optimal Ratio of Peptide to Azide Functionality:

The optimal ratio of peptide to azide functionality on mixed nanoparticles was first determined by screening the platelet recruitment ability of five different GRGDS: azide ratios, as shown herein: pure GRGDS-functionalized nanoparticles, 5:1, 3:1, 1:1, and pure azide-functionalized nanoparticles. This was performed to gauge whether or not the inclusion of a crosslinkable moiety resulted in increased platelet accumulation relative to the nanoparticle-only control, and to ensure that the click-functionalized groups and the lower percentage of platelet-targeting peptide did not adversely impact the ability of the nanoparticles to recruit platelets. Multiple rounds of incubation in platelet-rich-plasma (PRP) were performed to mimic the flow of fresh blood over the wound site, and this experiment was repeated with both four-arm-PEG-DBCO (4ADP) and DBCO-PEG-PLGA nanoparticles (DPP). All measurements were normalized to nonspecific binding to quiescent.

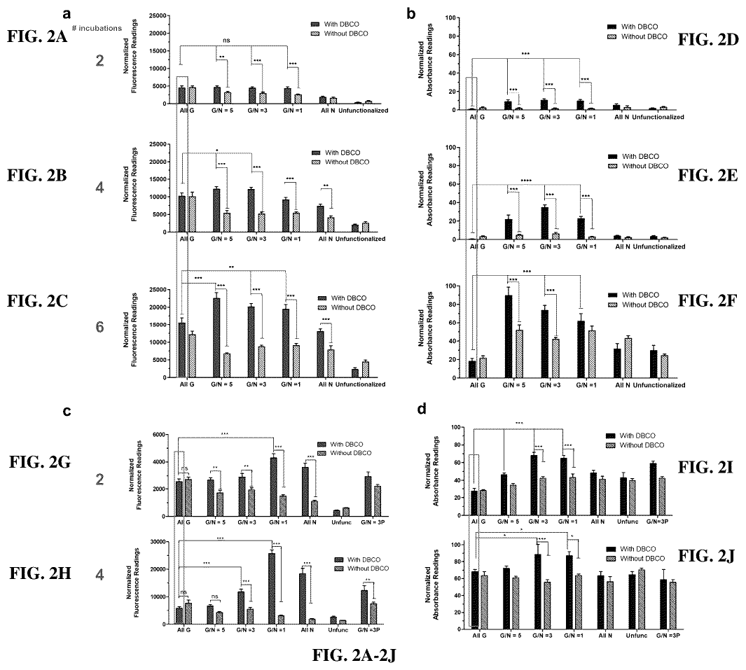

Increased platelet recruitment was observed at several ratios with the two-component system, occurring at the 4^{th} incubation for the combination of mixed GRGDS-azide nanoparticle (GNPP)+4ADP (FIG. 2B) and at the 2n d incubation for the combination of GNPP+DPP (FIG. 2D). Some degree of nonspecific binding or saturation of platelet binding was observed, in particular during the later incubation stages following multiple additions of platelet-rich-plasma and nanoparticle solution, phenomena that have been previously described in light transmission-based platelet aggregation assays and lactate-dehydrogenase-based binding assays. Increased nanoparticle recruitment was also observed for mixed nanoparticles (5:1 and 3:1 for GNPP+4ADP and 3:1 and 1:1 for GNPP+DPP). For both types of crosslinkers, a GRGDS-to-azide ratio of 3:1 resulted in significantly increased platelet recruitment relative to the nanoparticle-only control—as such, all future mixed nanoparticles were synthesized according to this ratio.

### IV. Methods of Using

A. Kits

The formulation is a two component kit, one containing the targeted nanoparticles and one containing the crosslinking agent. The kit may further include sterile saline or water for injection.

B. Dosages

In the examples where the formulation was administered to mice, a dosage of between 0.1 mg/mL and 5.0 mg/mL (e.g., 0.1 mg/mL and 2 mg/mL, 0.5 mg/mL and 1 mg/mL) was administered. These are just for the nanoparticle component. The polymeric crosslinker is delivered in a stoichiometric composition to the nanoparticle crosslinker. In the example where the formulation is administered to a pig, the nanoparticles were dosed at 1 and 2 mg/kg, and the polymers were 0.185 and 0.37 mg/kg (stoichiometric).

Studies were conducted where the dosage of hemostatic nanoparticles was decreased to confirm if a decreased dosage of the two-component system would achieve similar levels of platelet recruitment as a normal dose of nanoparticle-only treatment. Complement activation levels were also evaluated to gauge if decreasing the nanoparticle concentration could lead to lower complement production, as high levels of complement have been observed to exacerbate hemorrhage and can cause severe adverse side effects such as shock or even death. As the results demonstrate, the two-component system with both polymeric and nanoparticle crosslinker results in average platelet recruitment above that of nanoparticle-only groups at both lower concentrations tested. This was in part due to significantly lower nonspecific binding of the two-component system relative to the particle-only group, as evidenced by binding to quiescent platelets. Additionally, decreasing the dosage to 0.5 mg/mL in the two-component system with multi-arm polymer crosslinker resulted in an almost five-fold decrease (10.7% vs 51.2%) in Complement 5a (C5a) production compared to 1 mg/mL of the particle-only treatment, indicating that this system has the potential to decrease infusion reactions while retaining the hemostatic effects of particle-only treatments. Notably, the two-component system with nanoparticle treatment resulted in an increase in C5a concentration, an effect that could potentially be attributed to the formation of isolated aggregates that were less prevalent in the nanoparticle-polymer-crosslinker group.

In summary, crosslinking of the two components leading to nanoparticle aggregation was first confirmed to occur only at high concentrations and not at the concentration circulating in the bloodstream. The effect of this system on platelet recruitment over multiple incubations was then assessed and demonstrated to be superior to the nanoparticle-only system, and subsequent assays with different doses revealed that a lower dose of the two-component system could be used to achieve similar degrees of platelet recruitment to the nanoparticle-only system, resulting also in lower complement activation. The system was also able to recover platelet recruitment in diluted plasma to levels comparable to undiluted plasma with a nanoparticle-only treatment, as well as significantly improve clot formation relative to the nanoparticle-only group. When challenged with a liver resection injury model over the course of three hours, the two-component system resulted in a significant increase in survival relative to both saline and the particle-only control groups, corroborating the enhanced performance observed in vitro and presenting new avenues for the development of multicomponent hemostats.

C. Routes of Administration

The general purpose of this technology is to treat noncompressible hemorrhage and internal bleeding, which results in a significant percentage of preventable deaths in both military and civilian populations.

The nanoparticles are mixed with the cross-linking agents and administered by injection. These travel to the site of bleeding, where the nanoparticles bind to cells aggregating at the site of injury to form a hemostatic barrier.

The present invention will be further understood by reference to the following non-limiting examples. These demonstrate a two-component hemostat for targeted, bioorthogonal crosslinking was synthesized, tested and used for the treatment of internal bleeding by functionalization of PEG-b-PLGA nanoparticles with GRGDS and clickable azide moieties has been developed. Nanoparticles and multiarm polymers with corresponding copper-free crosslinking groups (DBCO) were delivered as a second component. The two-component system was demonstrated to only result in particle aggregation/significant concentration at several times circulating concentrations, and to enhance both platelet and nanoparticle recruitment at a peptide:azide ratio of 3:1. In addition to that, the system appeared to slow plasmin lysis of fibrin clots as monitored through a one-dimensional capillary assay, recover platelet recruitment and fibrin polymerization in hemodiluted environments, and require a lower therapeutic dose that resulted in decreased complement activation. Biodistribution, pharmacokinetics, and biocompatibility were evaluated in an uninjured model, and no signs of long-term inflammation or skewed biodistribution from the targeted nanoparticles were observed, confirming the safety of the system for systemic delivery. Finally, the two-component system was demonstrated to result in significantly improved survival relative to the nanoparticle-only group in a mouse liver resection model. These findings underscore the importance of supplementing the native hemostasis process in a comprehensive manner, providing guiding principles for the future development of artificial blood replacements.

### EXAMPLES

### Example 1: Synthesis and Validation of a Multi-Component System Including a Targeting Component and Crosslinking Component

A formulation was developed for the treatment of internal bleeding, with the ultimate goal of amplifying platelet recruitment and mitigating plasminolysis for greater clot stability.

### Methods

Materials

All chemicals and proteins were purchased from Sigma Aldrich unless otherwise specified. GRGDS peptide (95%) (SEQ ID NO:1) was purchased from China Peptides. RIPA Lysis Buffer, CyQUANT LDH Cytotoxicity Assays, Complement C5a Human ELISA kits were purchased from ThermoFisher Scientific; TNFα and IL-6 kits were purchased from Abcam. 4-arm-PEG-DBCO (20 kDa) was purchased from CreativePEGworks, and 4-arm-PEG-OH (20 kDa) was purchased from JenKem; both polymers were used as-is. Cyanine 7 free acid (Cy7) and N-Hydroxysuccinimide-DBCO was purchased from Lumiprobe. Deuterated solvents (CDCl₃ and DMSO-d₆) were purchased from Cambridge Isotope Laboratories. Citrated whole human blood was acquired from Research Blood Components (Watertown, MA) as a commercial, de-identified specimen, and donor consent was obtained via the company. Female BALB/c mice were purchased from Taconic.

Characterization Methods

All synthesized materials were characterized as previously described (Hong, et al., ACS Nano 2022, 16, 2494). In brief, polymers were characterized via nuclear magnetic resonance (NMR) on a Bruker Advance III DPX 400 spectrometer in deuterated DMSO at a concentration of 5 mg/mL. The ratio of lactide to glycolide was quantified via lactide protons (˜5.2 ppm) and glycolide protons (˜4.8 ppm), and the molecular weight of the polymer was calculated using the PEG macroinitiator as a standard (˜3.5 ppm). Mnova software was used to perform the analysis.

Gel permeation chromatography (GPC) was carried out at 1 mg/mL using an Agilent 1260 GPC system with three DMF ResiPore columns, a Wyatt Mini-DAWN TREOS 3-angle static light scattering detector, and a Wyatt Optilab T-rEX refractive index detector, with DMF+0.01M LiBr used as the eluent. Amino acid analysis was performed on an Agilent 1260 Infinity Quaternary LC System to detect peptide conjugation efficiency. Column conditions and elution gradients used were as previously described (Gkikas, et al., ACS biomaterials science & engineering 2019, 5, 2563). The polymer sample was dissolved in 6N HCl at a concentration of 10 mg/mL and hydrolyzed at 105° C. for 24 hours. Subsequently, excess HCl was removed via vacuum, and the residue was redissolved in 0.1 N HCl for analysis. A standard curve was generated for aspartic acid over the range of mM and used to calculate the amount of peptide conjugated to polymer (˜21 μmol GRGDS/g of PEG-b-PLGA).

Dynamic light scattering measurements were performed on Malvern Zetasizer Nano ZS90 in deionized water at 0.25 mg/mL. The laser wavelength on the machine was 633 nm. The acquisition angle was 90°. Acquisition times were set to automatic on the machine and averaged 60-70 seconds per run.

Polymer and Nanoparticle Synthesis

Polymers were synthesized and formulated for use with the multi-component system. The synthesis of each of the components Azide-Poly(ethylene glycol)-b-Poly(D,L-lactide-co-glycolide (N3-PEG-b-PLGA), DBCO-Poly(ethylene glycol)-b-Poly(D,L-lactide-co-glycolide (DBCO-PEG-b-PLGA), GRGDS-Poly(ethylene oxide)-b-Poly(D,L-lactide-co-glycolide) (GRGDS-PEG-PLGA), and Poly(ethylene oxide)-b-Poly(D,L-lactide-co-glycolide)-Cyanine 7 (PEG-PLGA-Cy7) is described in detail, below.

### (i) Azide-Poly(ethylene glycol)-b-Poly(D,L-lactide-co-glycolide (N3-PEG-b-PLGA)

450 mg of azide-PEG-OH (5 kDa, purchased from Sigma-Aldrich) was dissolved in 5 mL of dimethylformamide (DMF) and 5 mL of tetrahydrofuran (THF) and left overnight on activated molecular sieves. The solution was then transported to the glovebox, where 1,828 mg of racemic lactide was added to the solution and dissolved. The flask was then sealed with a rubber septum. In a separate glass vial, 1,120 mg of glycolide was dissolved in 4.5 mg of DMF and taken up in a syringe.

In another glass vial, 22 μL of 1,8-diazabicyclo(5.4.0)undec-7-ene (DBU) was mixed with 2 mL of DMF and also taken up in a syringe. The DBU solution was injected into the lactide and PEG solution; immediately afterwards, the glycolide solution was slowly infused into the flask via syringe pump at a rate of 60.4 μL per minute. The reaction was allowed to proceed for 90 minutes, before it was terminated via the addition of excess benzoic acid. The polymer was precipitated into isopropanol twice and diethyl ether once, resulting in a yield of 84% (˜2.5 g).

### (ii) DBCO-Poly(ethylene glycol)-b-Poly(D,L-lactide-co-glycolide (DBCO-PEG-b-PLGA)

DBCO-functionalized PEG-b-PLGA was synthesized via the use of DBCO-PEG macroinitiator. 100 mg of DBCO-N-hydroxysuccinimide ester (DBCO-NHS) was added to 1 g of NH2-PEG-OH (5 kDa) at a slight molar excess (˜1.16 equiv.), dissolved in 10 mL of anhydrous DCM, and stirred at RT overnight to react. The polymer solution was then concentrated to 5-6 ml and then precipitated in ether three times before it was dried under vacuum, resulting in a yield of 90% (90 mg). This macroinitiator was used in the same manner described in C. Hong, et al., ACS Nano 2022, 16, 2494. with no additional changes to solvent conditions or reaction time (80 minutes), with no additional changes to solvent conditions or reaction time.

### (iii) GRGDS-Poly(ethylene oxide)-b-Poly(D,L-lactide-co-glycolide) (GRGDS-PEG-PLGA)

GRGDS-PEG-b-PLGA was synthesized according to the following protocol. In brief, OH-PEG-NH2 was protected with Boc2O by incubating the polymer with Boc anhydride for three hours at room temperature and precipitating three times in ether. Following this step, the dried polymer was dissolved in dichloromethane (DCM) with lactide, while a glycolide solution in dimethylformamide (DMF) was prepared in a syringe, with a target molecular weight of ˜30 kDa PLGA. The reaction mixture was catalyzed via DBU and allowed to react for 80 minutes, over which the glycolide-DMF solution was infused via syringe pump. The polymer was precipitated twice in isopropanol and once in diethyl ether before it was dried under vacuum. Deprotection of the Boc protecting group was performed in 50% trifluoroacetic acid (TFA) and dichloromethane (DCM) overnight at room temperature, and the polymer was precipitated twice in diethyl ether after vacuum removal of TFA and DCM. Conjugation of the peptide was accomplished through a two-step process: activation of the amine terminus via carbonyldiimidazole (CDI) in dioxane at 37° C., followed immediately by incubation with a five-fold excess of GRGDS (SEQ ID NO:1) peptide over 48 hours. Excess peptide was then removed by dialyzing the solution for 72 hours in deionized water, after which the polymer was freeze-dried and stored at −20° C.

### (iv) Poly(ethylene oxide)-b-Poly(D,L-lactide-co-glycolide)-Cyanine 7 (PEG-PLGA-Cy7)

PEG-PLGA-Cy7 was synthesized according to the following protocol. In brief, one equivalent of Cyanine 7 free acid was added to PEG-b-PLGA-OH (synthesized using a methoxy-PEG macroinitiator) and dissolved in DCM with 0.4 equivalent of 4-dimethylaminopyridine (DMAP). N,N′-dicyclohexylcarbodiimide (DCC) was dissolved separately in DCM and added dropwise to the polymer solution under stir to achieve a final concentration of 50 mg polymer/mL solvent. The solution was stirred overnight at RT and precipitated in isopropanol and diethyl ether.

Nanoparticle Synthesis

Nanoparticles were synthesized through nanoprecipitation. In a first synthesis, 40 mg of PEG-b-PLGA polymer and 5 mg of Resomer 503H pure PLGA were dissolved in 1.5 mL dimethylformamide (DMF) and tetrahydrofuran (THF), stirred overnight, and sonicated until the solution was clear (˜15 min). The solution was then added dropwise into deionized water stirred at 720 rpm, before the resulting nanoparticle solution was washed repeatedly in Amicon ultracentrifugal filters with more deionized water to remove excess solvent and concentrate the solution. All nanoparticles were used within three days of purification, a timeframe over which they remained stable as measured via dynamic light scattering.

In a second synthesis, 35 mg of PEG-b-PLGA was added to 5 mg of pure PLGA (RESOMER® 503H, Sigma-Aldrich) and dissolved in 1.25 ml of tetrahydrofuran (THF) and 1.25 ml of dimethylformamide (DMF). This polymer solution was pipetted dropwise into deionized water stirred at 720 rpm at a solvent:water ratio of 1:5.

Different nanoparticles were synthesized by combining different types of PEG-PLGA polymer (PEG-b-PLGA, GRGDS-PEG-b-PLGA, N₃-PEG-b-PLGA, or DBCO-PEG-b-PLGA) in the solution. These nanoparticles were washed with deionized water in centrifugal filters with a molecular weight cutoff of 3 kDa and concentrated to a stock of 20 mg/mL, where they were stored at room temperature.

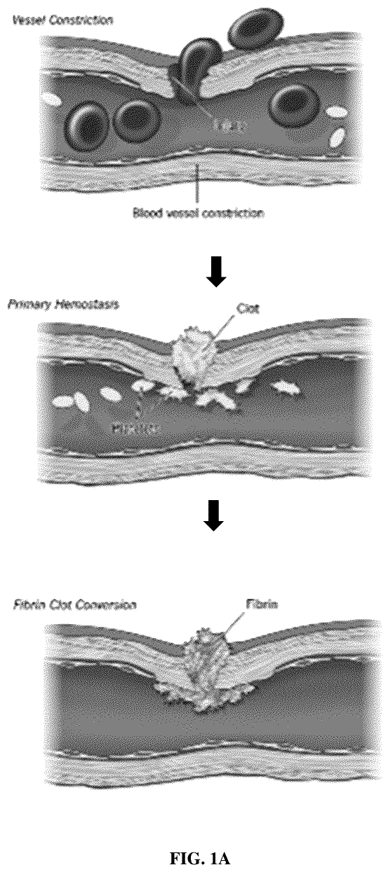

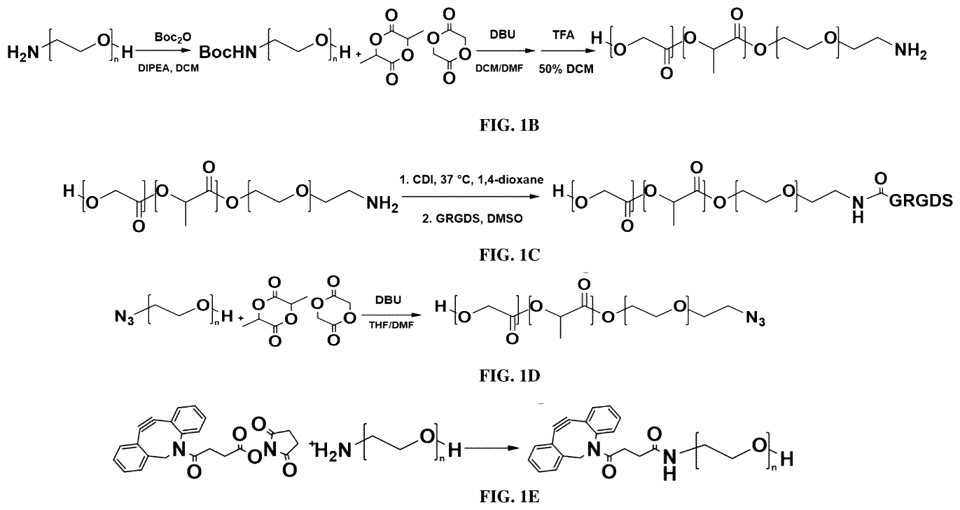

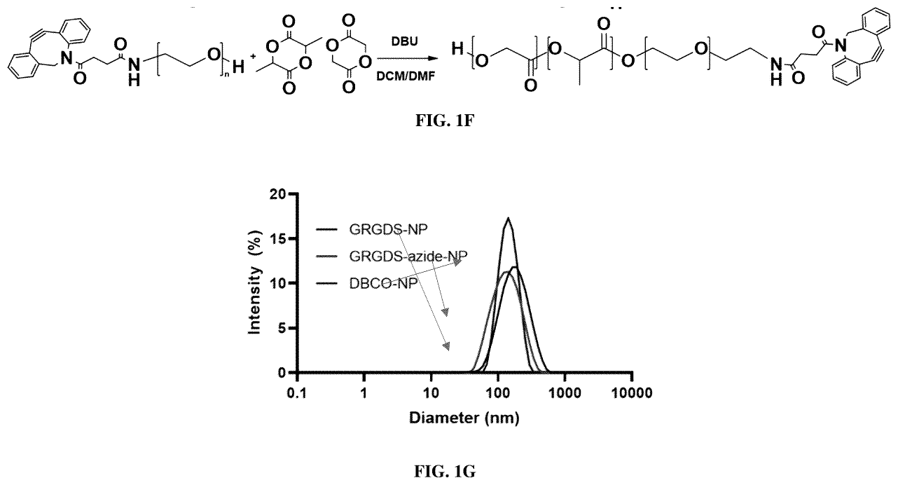

Mixed azide/GRGDS PEG-b-PLGA nanoparticles and crosslinking nanoparticles with dibenzylcyclooctyne (DBCO) moieties were synthesized via standard conjugation techniques and ring-opening polymerization (FIGS. 1A-1D). The functionality of the copper-free azide-DBCO click reaction was validated by tracking nanoparticle aggregation via dynamic light scattering relative to unfunctionalized controls. Multiple azide:GRGDS ratios were screened to determine the combination most suitable for in vivo experiments.

Characterization of Nanoparticle Crosslinking Via Dynamic Light Scattering

The crosslinking stability of click-functionalized nanoparticles was evaluated at concentrations of 2 mg/mL, 5 mg/mL, 10 mg/mL, 20 mg/mL, and 50 mg/mL. Circulating concentrations in animals were approximately 1-2 mg/mL. In brief, azide-functionalized nanoparticles were formulated using the synthesis conditions for the intermediate nanoparticle size, though the eventual size of the nanoparticles were slightly smaller—approximately 120 nm in diameter. DBCO-functionalized nanoparticles and methoxy-functionalized nanoparticles were similarly synthesized, as were mixed nanoparticles with varying ratios of GRGDS peptide (SEQ ID NO:1) to azide functionality. Four-arm-DBCO-functionalized PEG (4ADP, MW=20 kDa) and four-arm unfunctionalized PEG (4AP, MW=20 kDa) was dissolved to stoichiometric concentrations calculated from the molecular weights of the azide-functionalized polymer. For a purely azide-functionalized polymer (˜27 kDa) nanoparticle at 1 mg/mL in deionized water, this would be equivalent to 0.1852 mg/mL 4ADP (20 kDa). The formula (Formula I) for obtaining this is shown below.

$${Concentration}_{4 ADP} = \frac{{Concentration}_{azide}}{{MW}_{azide - PEG - PLGA}} \times \frac{{MW}_{4 ADP}}{4} \qquad Formula I$$

For each concentration previously specified, 25 μL of azide-functionalized nanoparticle at twice the desired concentration was added to a PCR tube. 25 μL of DBCO-functionalized nanoparticle was added to this tube. This step was repeated with 25 μL of unfunctionalized nanoparticle, μL of four-arm-PEG-DBCO, and 25 μL of unfunctionalized four-arm-PEG in three different PCR tubes. These tubes were incubated at 37 deg Celsius with a water basin to prevent evaporation, and these were measured at the start of the experiment to record the size at the start of the experiment. Samples were taken at 2 h, 4 h, and 24 h post-incubation to track size changes in the nanoparticles, using the unfunctionalized combinations as negative controls.

Blood Components Preparation

Citrated whole blood was separated into hematocrit and platelet-rich-plasma (PRP) upon receipt. In brief, blood was centrifuged at 200 rcf in a 50 mL conical tube for 20 minutes with no brake at room temperature. The upper fraction, PRP, was then used in subsequent ex vivo assays (platelet recruitment and transmission-based fibrin polymerization assays).

In Vitro/Ex Vivo Evaluation of Hemostatic Efficacy

### (i) Determining the Optimal Peptide to Azide Ratio Using a Platelet Recruitment Assay

The effect of nanoparticle size on the total amount of platelet aggregation/binding to a surface was evaluated by a lactate dehydrogenase (LDH) assay. In brief, a mixture of platelet-rich-plasma and nanoparticle solution was added to black cell-treated wells (54 μL PRP to 6 μL nanoparticle stock solution at 11 mg/mL). Half the wells received 6 μL adenosine diphosphate stock solution (ADP) as a platelet activation agonist, while the other half received saline. Four-arm-PEG-DBCO and DBCO-PEG-PLGA nanoparticles were added in a 6 μL dose at stoichiometric equivalence to the azide-functionalized nanoparticles. The mixture was allowed to incubate for an hour before it was washed three times with saline (100 μL per well). The prior few steps (addition of PRP, nanoparticle stock, crosslinker/saline, and agonist/saline) were then repeated for following incubation steps. After the desired number of incubations, the wells were again washed three times with saline before 60 μL of Pierce RIPA lysis buffer was added. These samples were then diluted threefold and then used in the CyQuant LDH assay according to manufacturer's instructions in a clear-bottom well plate. The plate's absorbance was then measured at 490 nm and 680 nm, with the final results calculated by subtracting the readings at 680 nm for those at 490 nm. Six incubations were completed for the four-arm-PEG-DBCO system, and four incubations were completed for the DBCO-PEG-PLGA system. Each trial group included six replicates with the exception of outliers as excluded via Grubbs' test.

### (ii) Evaluating Platelet Recruitment at Lower Nanoparticle Doses

A variation of the prior assay using different nanoparticle doses was later completed with four incubations. Nanoparticle stock solutions were formulated at 11 mg/mL, 5.5 mg/mL, and 2.75 mg/mL and diluted in PRP as described to attain final concentrations of 1 mg/mL, 0.5 mg/mL, and 0.25 mg/mL, respectively. Only 0.5 and 0.25 mg/mL concentrations of DBCO-NP crosslinkers were tested to keep total nanoparticle concentration below 1 mg/mL, as complement activation was observed at higher nanoparticle concentrations. All subsequent steps are as described in the prior section. Each trial group included six replicates with the exception of outliers as excluded via Grubbs' test.

### (iii) Evaluating Complement Activation of Hemostatic Nanoparticles

Complement activation of hemostatic nanoparticles was measured following a protocol modified from Maisha, et al., Nano Letters 2021, 21, 9069. In brief, PRP was incubated with nanoparticle solutions or zymosan (positive control, 0.5 mg/mL) at concentrations of 0.5-1 mg/mL in isotonic glucose for 45 minutes at 37° C. under agitation. They were then spun down at 3000×g to remove nanoparticles and platelets. The resulting clear serum was then assayed via Invitrogen's Complement C5a Human ELISA Kit according to manufacturer's instructions at a dilution of 1:25 (kit included pre-coated ELISA wells). All reported results were normalized to C5a concentrations in a blank isotonic glucose control.

### (iv) Evaluating Platelet Recruitment in Hemodiluted Conditions

A variation of the platelet recruitment assay was again performed, this time with various dilutions of plasma. In brief, plasma was diluted to 60% and 80% its original concentration by combining 30 mL of PRP with mL of isotonic saline or 40 mL of PRP with 10 mL of isotonic saline, respectively. These were denoted as −40% −20%, and 0% dilution, respectively. The PRP dilutions and undiluted PRP were all tested with the two-component system and nanoparticle-only groups at 0.5 mg polymer/mL PRP. All subsequent steps are as described in the section ‘Determining the optimal peptide to azide ratio using a platelet recruitment assay’. Each trial group included six replicates.

### (v) Evaluating Fibrin Crosslinking in Hemodiluted/Dilutional Coagulopathy Conditions

Fibrin crosslinking under dilutional coagulopathy conditions was evaluated using platelet-rich-plasma diluted to 60% its original concentration to simulate Grade IV hemorrhage. In brief, 7.5 μL of nanoparticle solutions at 44 mg/mL or deionized water, 6 μL of CaCl2, and 52.5 μL of diluted PRP was added to each well of a clear flat-bottom non-coated 96-well plate to obtain a final concentration of 5 mg nanoparticle/mL PRP. The change in absorbance over time was then monitored at 650 nm at one-minute intervals for one hour. All absorbance or transmission measurements were normalized to initial values at t=0 min to account for variations due to nanoparticle scattering. The overall coagulation potential, OCP, was calculated by plotting the normalized absorbance curves and computing the area underneath each curve; the change in transmission was calculated by converting absorbance to transmission via the following equation: Transmission=10^{2-Absorbance} and subtracting the initial transmission value. Twelve replicates were collected for this measurement.

### (vi) Evaluating Fibrin Clot Lysis in Hemodiluted/Dilutional Coagulopathy Conditions

A 1-dimensional capillary degradation assay was developed in order to assess the effect of the two-component system on the stability of fibrin clots when exposed to plasma. 50 mg of human fibrinogen (plasminogen-free) was dissolved in 5 mL HEPES buffered saline and labeled using AF488-N-hydroxysuccinimide (AF488-NHS). This was washed multiple times using an Amicon ultracentrifugal filter or dialyzed overnight to remove unreacted dye before it was diluted to a stock concentration of 5.5 mg/mL. Square glass capillaries of 1.0 mm inner diameter were ordered from VitroCom. 250 μL of fibrinogen solution was added to 250 μL nanoparticle stock solution and 100 μL HEPES buffered saline or 100 μL multiarm polymer/crosslinker solution and mixed well. 30 μL of thrombin (100 U/mL) and 500 μL HEPES buffered saline was added to a glass shell vial and mixed thoroughly. The fibrinogen mix (˜1.25 mg/mL) was then pipetted into the glass shell vial and the capillaries were immediately immersed in the gelling mixture for 45 minutes. One end of the capillary was sealed with vacuum grease. Plasmin was then added using a gel loading pipette tip directly at the capillary interface until the capillary was full, at which point it was also sealed with vacuum grease. The capillaries were then imaged on a fluorescent microscope for a period of 24 hours to track the degradation of the fibrin clot, which was measured by the increase in fluorescence due to fibrinogen loss on the plasmin end of the capillary.

### Results

Nanoparticle Synthesis

PEG-b-PLGA copolymers functionalized with platelet-binding peptide GRGDS (SEQ ID NO:1), DBCO, Cyanine 7, or azide groups were synthesized via ring-opening polymerization as previously described by Hong, et al., Biomaterials 2022, 283, 121432, with minor adjustments in solvent and reaction time made to accommodate different functional groups. These polymers were used to generate nanoparticles (NPs) of approximately 180 nm, as previous studies had demonstrated that this size was optimal for the recruitment of activated platelets and accumulation at the injury site. With the exception of pure azide NPs (only used in kinetic studies), all sizes fell within the range of 140-220 nm. Cyanine 7 (Cy7)-labeled nanoparticles were synthesized via mixing PEG-b-PLGA-Cy7 with GRGDS-PEG-b-PLGA and using the polymer solution for nanoprecipitation, while mixed azide-GRGDS nanoparticles (GNPP) were likewise synthesized through mixing of the two polymers and subsequent nanoprecipitation. DBCO-PEG-b-PLGA nanoparticles were also synthesized through nanoprecipitation, and 4-arm-DBCO PEG was purchased and used as-is. For ease of comparison to the single-component system, the concentration of the two-component system refers to nanoparticle concentration unless otherwise specified. FIGS. 1A-D provide the synthetic scheme of all functionalized PEG-b-PLGA polymers and representative nanoparticle size distributions as measured via dynamic light scattering (DLS) (FIG. 1E). NP diameters are provided in Table 1, below.

TABLE 1
Nanoparticle (NP)sizes as measured via DLS.

| | |
|---|---|
| Diameter (Z-average) | PDI |

| | | | |
|---|---|---|---|
| GRGDS-azide NPs | 182.2 ± 1.04 | nm | 0.226 |
| GRGDS-NPs | 172.7 ± 3.9 | nm | 0.235 |
| DBCO-NPs | 143.0 ± 2.58 | nm | 0.214 |
| Azide-NPs | 96.8 ± 0.18 | nm | 0.184 |

Optimal Ratio of Peptide to Azide Functionality

The optimal ratio of peptide to azide functionality on mixed nanoparticles was first determined by screening the platelet recruitment ability of five different GRGDS:azide ratios, as shown herein: pure GRGDS-functionalized nanoparticles, 5:1, 3:1, 1:1, and pure azide-functionalized nanoparticles. This was performed through a lactate dehydrogenase (LDH) assay to gauge whether or not the inclusion of a crosslinkable moiety resulted in increased platelet accumulation relative to the nanoparticle-only control, and to ensure that the click-functionalized groups and the lower percentage of platelet-targeting peptide did not adversely impact the ability of the nanoparticles to recruit platelets. Multiple rounds of incubation in platelet-rich-plasma (PRP) were performed to mimic the flow of fresh blood over the wound site, and this experiment was repeated with both four-arm-PEG-DBCO (4ADP) and DBCO-PEG-PLGA nanoparticles (DPP). All measurements were normalized to nonspecific binding to quiescent platelets through the lactate dehydrogenase (LDH) assay.

Increased nanoparticle (FIGS. 2A-2C) and platelet recruitment was observed at several ratios with the two-component system, occurring at the 4th incubation for the combination of mixed GRGDS-azide nanoparticle (GNPP)+4ADP (FIGS. 2D-2F) and at the 2nd incubation for the combination of GNPP+DPP (FIGS. 2I-2J). Some degree of nonspecific binding or saturation of platelet binding was observed, in particular during the later incubation stages following multiple additions of platelet-rich-plasma and nanoparticle solution, phenomena that have been described in light transmission-based platelet aggregation assays and lactate-dehydrogenase-based binding assays. Increased nanoparticle recruitment was also observed for mixed nanoparticles (5:1 and 3:1 for GNPP+4ADP and 3:1 and 1:1 for GNPP+DPP). For both types of crosslinkers, a GRGDS-to-azide ratio of 3:1 resulted in significantly increased platelet recruitment relative to the nanoparticle-only control—as such, all future mixed nanoparticles were synthesized according to this ratio.

Crosslinking Behavior of the Two-Component System

As wound-targeting nanoparticles were observed in several studies to result in significantly higher accumulation at the wound site (Lashof-Sullivan, et al., ACS biomaterials science & engineering 2016, 2, 385), the aim of the two-component system was to leverage this phenomenon and promote significant crosslinking selectively at higher concentrations. The formation of a gel occurs only when polymer concentrations exceed the critical gelation concentration (CGC) (Pérez-Rentero, et al., Applied Sciences, 10.3390/app8050671), which has been reported at concentrations of 130 mg/mL for PEG-PLGA and 260 mg/mL for PEG-PLA gels (Jeong Lee, et al., Polymer Journal 2009, 41, 425; Li, et al., Langmuir 2007, 23, 2778). Representative inversion tests of the nanoparticle solutions at 150 mg/mL at 1:1 stoichiometry confirmed gelation within this range.

To ensure safe intravenous delivery of the two-component system, nanoparticle concentrations ranging from 2 mg/mL up to 50 mg/mL were incubated with crosslinkers at a 1:1 stoichiometric ratio, between the circulation concentration of 0.5-1 mg/mL and the CGC. FIGS. 3A-3H depict the behavior of the two-component system crosslinking at various concentrations with Pure azide nanoparticles (FIGS. 3A-3D), and Azide-GRGDS nanoparticles (FIGS. 3E-3H), respectively.

Using dynamic light scattering to monitor crosslinking behavior, the functionality of the clickable groups was first evaluated using pure azide nanoparticles (FIGS. 3A-3D), before mixed nanoparticles were also tested at these concentrations (FIGS. 3E-3H). Representative inversion tests of the nanoparticle solutions at 200 mg/mL have also been provided in FIGS. 3I-3J. Control groups of unfunctionalized nanoparticle (MPP) and unfunctionalized four-arm-PEG (4AUP) were included to confirm that any observed aggregation did not occur due to high nanoparticle concentration and subsequent sedimentation.

Pure azide-functionalized nanoparticles (FIG. 3A-3D) demonstrated visible increases in size within the first two hours of the experiment when incubated with both nanoparticle and multiarm polymer crosslinkers at 50 mg/mL. No significant increases in sizes were observed at 5-10 times circulating concentration (0.5-1 mg/mL) for the nanoparticle+nanoparticle crosslinker combination (NPP+DPP), while a slight increase in size was observed in the nanoparticle+multiarm polymer system (NPP+4ADP). No size increases were observed with the control groups of unfunctionalized nanoparticle and unfunctionalized multiarm polymer. Mixed nanoparticles (GNPP) were also observed to increase in size at higher concentrations, though the kinetics appeared to be delayed in comparison to the pure azide nanoparticles, likely due to the lower percentage of cross-linkable functionalities (FIGS. 3E-3H). Two types of DBCO-functionalized nanoparticle crosslinkers were tested in this experiment—one of pure DBCO-PEG-b-PLGA (DPP), and one mixed with GRGDS-PEG-b-PLGA (GDPP). Nanoparticle aggregation behavior was observed to be much more pronounced in the GNPP+DPP solution despite the same amount of DBCO-functionalized polymer, a phenomenon that could be potentially attributed to the higher functionality per molecule of pure DBCO-functionalized nanoparticles. As a result, all further experiments were conducted with DPP instead of GDPP, to ensure crosslinking could still occur upon accumulation at the injury site. Only minimal increases in nanoparticle size were observed for the GNPP+4ADP combination at circulating concentration, and no increases in size were observed with unfunctionalized nanoparticles. Overall, the two-component system with either nanoparticle or multiarm polymer crosslinker led to significant nanoparticle aggregation at higher concentrations but not at circulating concentration.

Platelet Recruitment at Decreased Doses of Hemostatic Nanoparticle

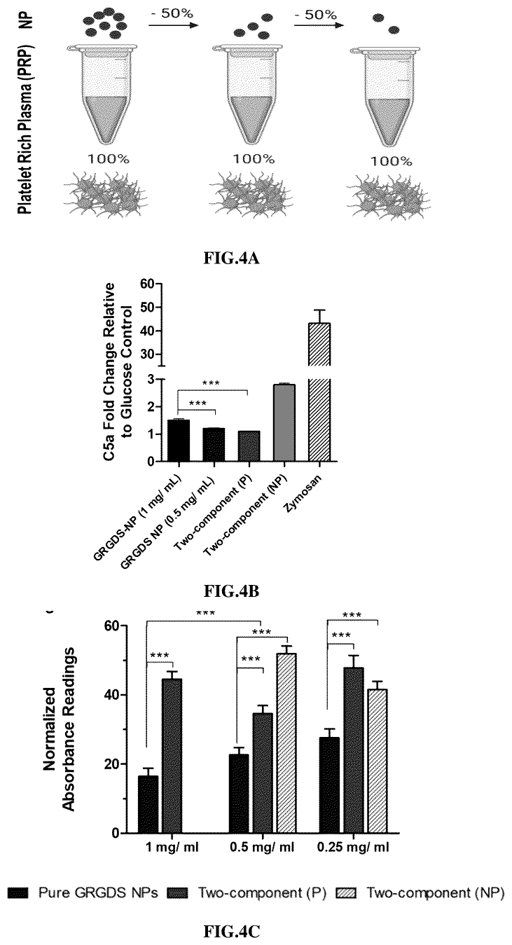

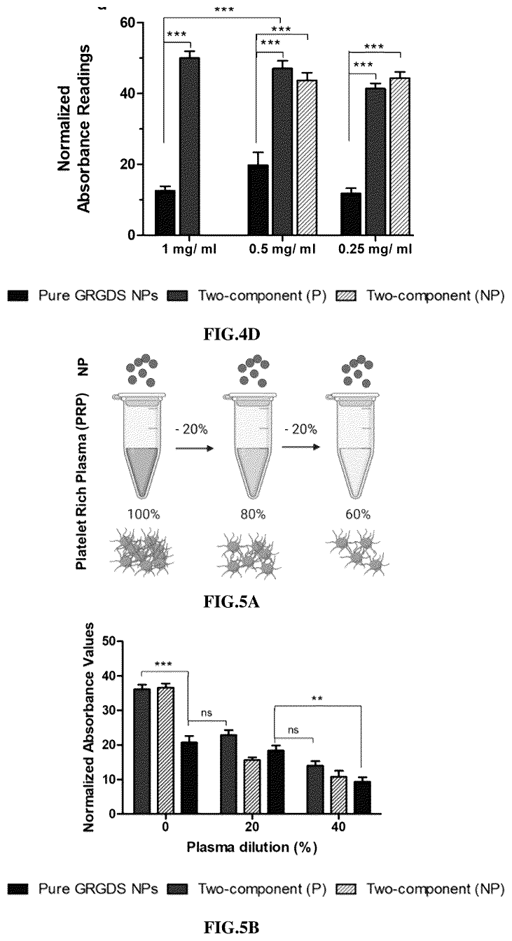

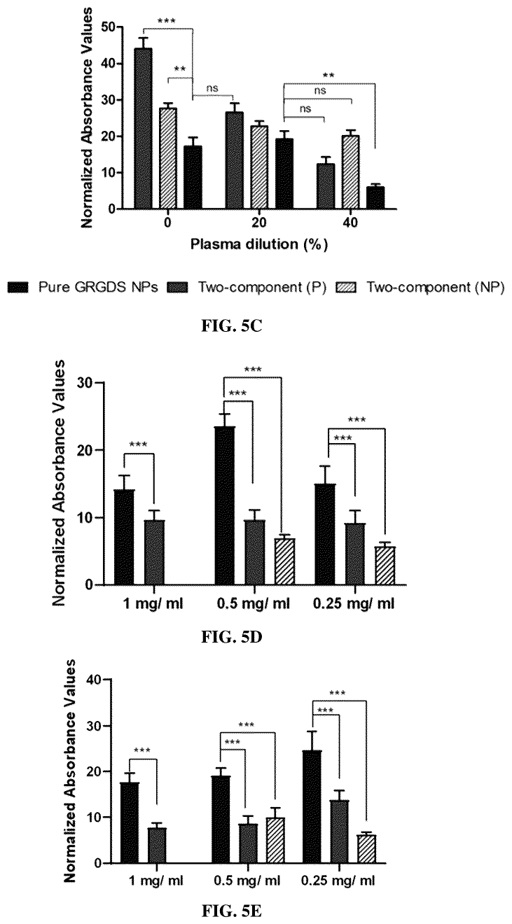

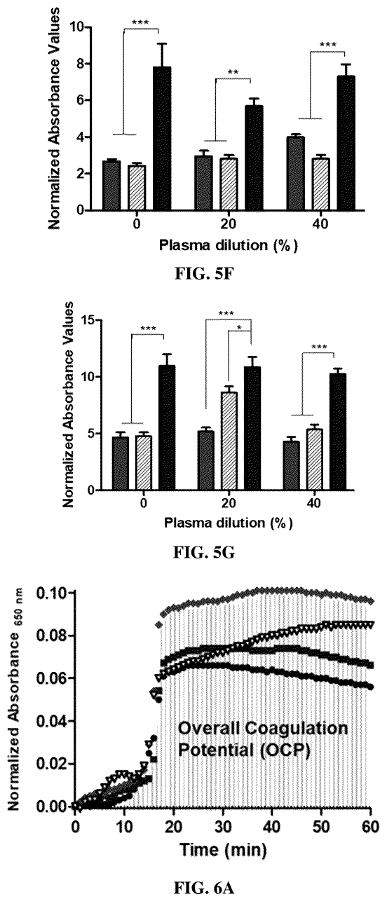

The dosage of hemostatic nanoparticles was then decreased to confirm if a decreased dosage of the two-component system would achieve similar levels of platelet recruitment as a normal dose of nanoparticle-only treatment (FIGS. 4A-4D). Complement activation levels were likewise evaluated to gauge if decreasing the nanoparticle concentration could lead to lower complement production, as high levels of complement have been observed to exacerbate hemorrhage and can cause severe adverse side effects such as shock or even death. As can be seen, the two-component system with both polymeric and nanoparticle crosslinker results in average platelet recruitment above that of nanoparticle-only groups at both lower concentrations tested. This was in part due to significantly lower nonspecific binding of the two-component system relative to the particle-only group, as evidenced by binding to quiescent platelets (FIG. 5D). Additionally, decreasing the dosage to 0.5 mg/mL of nanoparticle in the two-component system with multiarm polymer crosslinker resulted in an almost five-fold decrease (10.7% vs 51.2%) in Complement 5a (C5a) production compared to 1 mg/mL of the particle-only treatment, suggesting that this system has the potential to decrease immune-mediated side effects while retaining the hemostatic effects of particle-only treatments. In contrast, the two-component system with nanoparticle treatment resulted in an increase in C5a concentration, an effect that could potentially be attributed to the formation of isolated aggregates (La-Beck, et al., Frontiers in immunology 2020, 11, 603039). that were less prevalent in the nanoparticle-polymer-crosslinker group.

Platelet Recruitment Under Dilutional Coagulopathy Conditions

Subsequently, the effect of the two-component system on recovering platelet recruitment in hemodiluted environments was evaluated. In brief, PRP was diluted by 20% and 40% with isotonic saline, simulating Grade II-Grade IV blood loss with subsequent fluid resuscitation (FIGS. 5A-5D). The two-component system was observed to significantly enhance platelet recruitment in comparison to the single-component system at these diluted concentrations for both two incubations and four incubations. In addition to that, the system was observed to recover platelet recruitment to a level equivalent to 20% less dilution (e.g., 20% dilution vs. undiluted plasma or 40% dilution vs. 20% dilution), demonstrating the improved functionality of the two-component system relative to the single-particle system even in hemodiluted environments with decreased fibrinogen, platelet, and clotting factors.

Fibrin Clot Formation and Degradation Under Severe Dilutional Coagulopathy Conditions

The two-component system was found to significantly improve clot formation and decrease clot degradation under severe dilutional coagulopathic conditions. In brief, PRP was isolated from citrated whole blood and diluted by 40% in isotonic saline, whereupon it was incubated with trial groups of saline, GRGDS-NPs, and both polymeric and nanoparticle versions of the two-component at a concentration of 5 mg/mL nanoparticle. This concentration—5 to 10-fold the circulating concentration of 0.5-1 mg/mL—was used in the following experiments, as prior studies had demonstrated that the hemostatic nanoparticles accumulated at the injured vessel section at approximately 5 to 15 times relative to uninjured vessels, though it is possible that concentrations localized at the point of hemorrhage could exceed even that. The absorbance of these solutions following the addition of CaCl2 was then monitored to yield the overall coagulation potential (OCP) and change in transmission as a measure of fibrin polymerization (FIGS. 6A, 6C).

As can be seen, the two-component system is capable of significantly increasing fibrin polymerization by up to 149% even in severe dilutional coagulopathic conditions relative to the saline control (FIGS. 6B, 6D). In particular, the two-component system with polymeric crosslinker also significantly enhanced clot formation relative to the nanoparticle-only control (132% and 125% for OCP and change in transmission, respectively). While similar anticoagulated or coagulopathic models have been proposed in literature to evaluate the effect of synthetic hemostats on clot formation, they have generally only depleted or inhibited specific components of hemostasis, such as platelets, coagulation factors, or fibrinogen (Chan, et al., Science Translational Medicine 2015, 7, 277ra29; Girish, et al., ACS Nano 2022; Sekhon, et al., Science Translational Medicine, 14, eabb8975). Direct dilution of platelet-rich-plasma with saline instead of platelet-poor-plasma results in the decrease of all plasma components, which is observed upon fluid resucitation in response to massive hemorrhage (Ishikura, et al., UpToDate 2020; Fries, et al., AINS 2004, 39, 745). Overall, the two-component system shows promise in recovering fibrin polymerization, which is critical to achieving hemostasis in traumatic bleeding.

Subsequently, fibrin clot degradation was assessed using a one-dimensional degradation assay in a glass capillary, as fibrinolysis is likewise exacerbated in dilutional coagulopathy (Bolliger, et al., Anesthesiology 2010, 113, 1205). Fluorescently-labeled fibrinogen was gelled with thrombin at 1.25 mg/mL in a capillary, this time mimicking severely coagulopathic conditions below critical fibrinogen thresholds with complete depletion of platelets and coagulation factors (Chan, et al., Science Translational Medicine 2015, 7, 277ra29). This was then filled with plasmin using a gel loading tip, and fibrinogen degradation was measured via diffusion of the labeled fibrinogen into the plasmin solution, as illustrated in FIG. 6E.

The inclusion of the single-component hemostatic nanoparticles led to a slight decrease (˜25%) in the level of fibrinogen diffusion into the plasmin chamber over the course of 24 hours, while the addition of the two-component system led to approximately a 40% decrease in fibrinogen loss. FIG. 6F illustrates the degradation profiles of fibrin clots in nanoparticle-free, hemostatic nanoparticle, and two-component system trial groups, where fibrinolysis has been mitigated over time. In conjunction with the enhancements in fibrin polymerization and platelet recruitment, these results demonstrate the significant benefit of wound-targeted crosslinking to promote clot formation under coagulopathic conditions.

### Example 2: Biodistribution, Pharmacokinetics, and Biocompatibility in an Uninjured Mouse Model

### Methods

Biodistribution, Biocompatibility, and Pharmacokinetics:

Female BALB/c mice (12-15 weeks old, 20-30 g) were used in accordance with procedures approved by the MIT Division of Comparative Medicine and the Institutional Animal Care and Use Committee (IACUC) of MIT Animals had ad libitum access to both food and water until the time of the procedure. Mice were injected via tail vein with the two-component system (hemostatic nanoparticle and nanoparticle crosslinker), with the two injections spaced five minutes apart. The nanoparticles were labeled with Cy7 to facilitate imaging via In Vivo Imaging System (IVIS) at Ex. 745 nm/Em. 800 nm. Trial groups included nanoparticle+saline, unfunctionalized nanoparticle+hemostatic nanoparticle, various concentrations of crosslinking polymer, and a 1:1 stoichiometric ratio of crosslinking nanoparticle to hemostatic nanoparticle. These mice were imaged at set intervals over a period of 2.5-3 hours, and then sacrificed to obtain the organ biodistribution of hemostatic nanoparticles. Blood was drawn from the cheek at the start and at the end of the experiment to evaluate the circulating concentration of the different trial groups. A small cohort of mice was monitored over the next few months for any adverse effects; cheek bleeding was performed at two weeks post-injection. TNFα and IL-6 levels were determined through ELISA according to manufacturer's instructions.

### Results

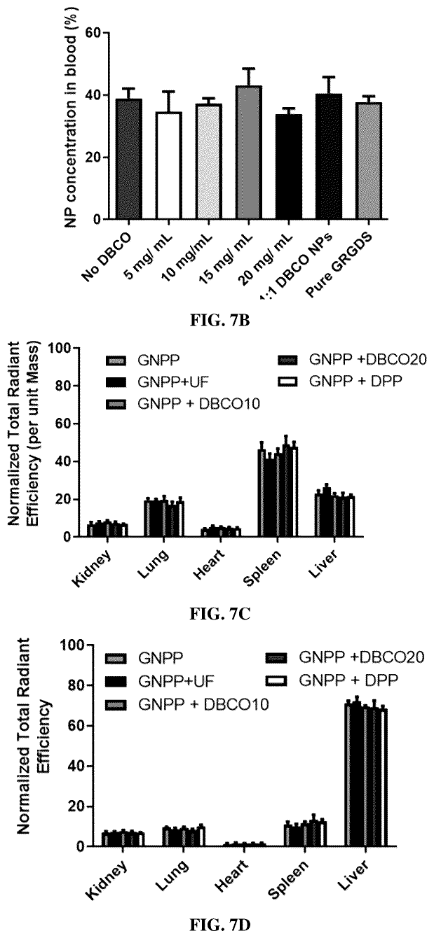

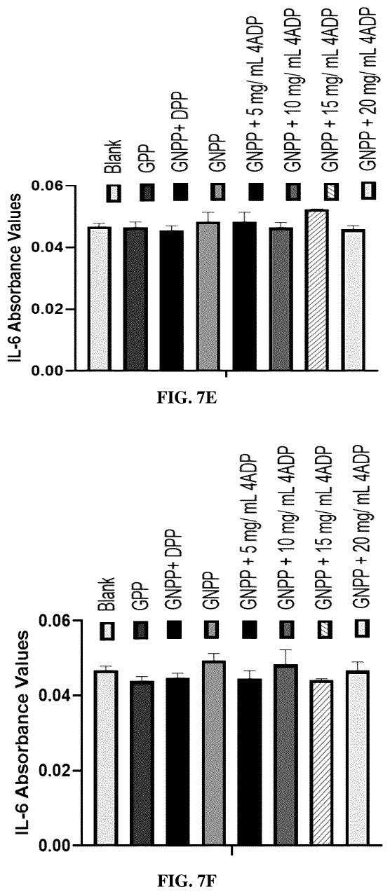

To ascertain the safety of the system, the biodistribution, pharmacokinetics, and biocompatibility in an uninjured mouse model were first measured using Cy7-labeled fluorescent nanoparticles. The effect of DBCO crosslinker concentration on retention and blood circulation lifetime was also assessed. As shown in FIGS. 7A-7F, there appears to be a slight decrease in retention time for higher concentrations of polymeric crosslinker, though this was not found to be significant. Notably, this was not observed in the two-component system with nanoparticle crosslinker. No differences in nanoparticle circulating concentration could be detected among the trial groups (FIG. 7B), and no visible differences in accumulation could be observed via IVIS images. These mice were monitored for two weeks post-injection for any long-term inflammation due to the injection through the measurement of TNFα and IL-6 levels, cytokines commonly used to indicate the presence of inflammation (Oike, et al., Scientific Reports 2018, 8, 15783; Litman, et al., Scientific Reports 2021, 11, 17535). No significant differences were observed between trial groups and mice that had not been injected (FIGS. 7E-7F), indicating that the two-component system does not pose any additional risk for long-term inflammation despite its additional crosslinking ability. This may be because at low concentrations any reaction between the two components is incapable of forming large aggregates, as shown in vitro in FIGS. 3A-3J.

Similarly, organ biodistribution was found to remain similar over all groups tested, with the highest concentrations of DBCO crosslinker selected from the previous study to visualize any potential differences between groups. This indicates that despite the higher degree of kidney clearance of polymers (relative to nanoparticles) (Veronese, et al., Bioconjugate Chem. 2005, 16, 775), the non-targeted crosslinkers did not significantly skew the biodistribution of the peptide-functionalized, targeted nanoparticles. Additionally, no significant increases in pulmonary accumulation were observed in the two-component system, corroborating the prior in vitro results that demonstrated no significant nanoparticle aggregation at circulating conditions.

### Example 3: Hemostatic Efficacy of the Two-Component in a Liver Resection Mouse Model

Methods

Lethal Liver Resection Model

Female BALB/c mice (20-30 g) were injected via tail vein with the two-component system (hemostatic nanoparticle and nanoparticle crosslinker), with the two injections spaced five minutes apart. The mouse was anesthetized via inhaled isoflurane at 2.0-2.5%, and depth of anesthesia assessed with toe pinch. The mouse was then positioned supine on a surgical board and its four limbs secured with tape, with a heating pad placed beneath the mouse. The abdomen and groin were shaved and wiped down with betadine. A tube containing 0.5 ml of PBS, two pre-weighed gauze pieces, and one weigh boat was prepared. The liver was exposed via a ventral midline laparotomy incision, and one piece of gauze was placed on either side of the abdominal cavity. A ˜5 mm section of the left-middle lobe of the liver was removed with scissors, and this section was placed in the tube with PBS. The incision was then closed with wound clips. The mouse was observed under anesthesia for a period of <3 hours and euthanized at 3 hours via cardiac puncture or isoflurane overdose (e.g., 5% isoflurane for 5 mins and vital organ removal/exsanguination.) Blood loss was determined by weighing gauze immediately following conclusion of the experiment, and organs was weighed and imaged via IVIS to obtain organ biodistribution from fluorescently labeled nanoparticles.

Results

The hemostatic efficacy of the two-component system was evaluated in a closed liver resection model, with an observation period of three hours. BALB/c mice were dosed with prophylactic injections of the two-component system, as vasoconstriction due to acute hemorrhage led to challenges in successful tail-vein injections. Two doses of nanoparticle-only treatment were administered: the original concentration at 1 mg/mL, which was previously observed to increase complement activation by ˜50% relative to the particle-free concentration (FIGS. 5A-5G), and the decreased dose at 0.5 mg/mL for comparison with the two-component system at the same nanoparticle concentration. Three mice per group were injected with Cy7-labeled nanoparticles and dissected to obtain organ biodistribution.

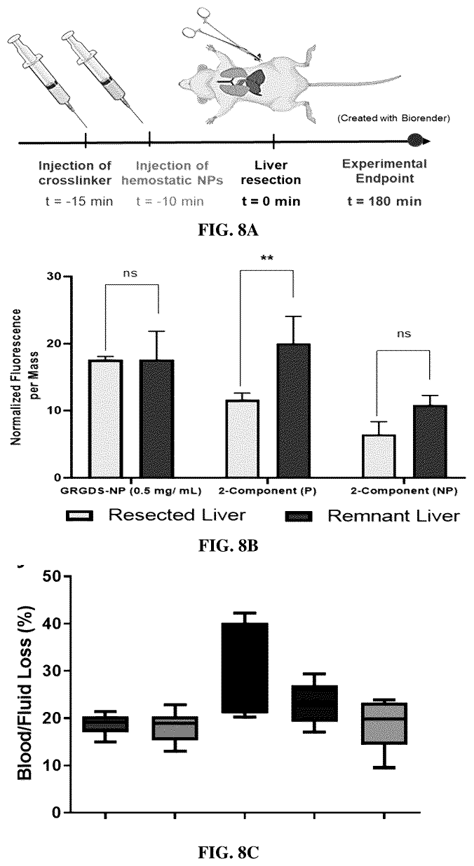

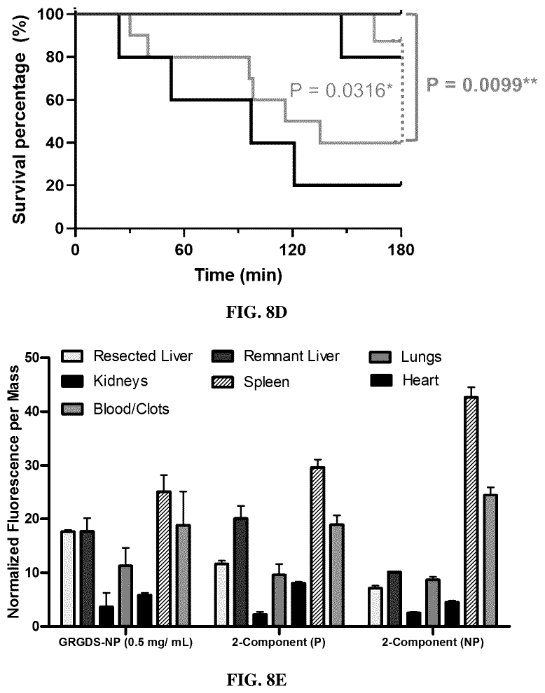

As can be seen in FIGS. 8A-8E, both the two-component systems with polymeric and nanoparticle crosslinkers resulted in significantly increased survival (100% survival at P=0.0099** and 87.5% survival at P=0.0316*, respectively). Though prophylactic treatment may have contributed to increased efficacy, these results are relative to the nanoparticle-only group at the same dose (40% survival), as well as the control. While no significant differences in blood loss were observed between these trial groups, this phenomenon is consistent with similar studies on uncontrolled, lethal hemorrhage models (Gao, et al., Science Advances 2020, 6, eaba0588). The two-component system also resulted in a significant difference in remnant vs. resected liver accumulation, which was not observed in the nanoparticle-only system, potentially indicating increased accumulation at the injury site. The two-component system with nanoparticle crosslinker likewise exhibited a similar trend in enhanced liver accumulation. As no significant differences in blood fluorescence levels were detected (FIG. 8E), this suggests that the difference in accumulation is not entirely due to a higher level of hepatic clearance over time, as the concentration of nanoparticles in blood should have also decreased. This corroborates ex vivo results obtained in FIGS. 2A-2J, where increased nanoparticle and platelet accumulation was observed upon the inclusion of a crosslinking component to the nanoparticle-only system. The biodistribution profiles of other organs have been provided in FIG. 8E.

The results of the injury model corroborate conclusions drawn from prior in vitro and ex vivo experiments regarding the efficacy of the two-component system. Specifically, the two-component system with polymer crosslinker overall achieved the most significant improvement in platelet recruitment both under normal and hemodiluted conditions, in decreasing complement activation, and in fibrin polymerization relative to the nanoparticle-only treatment group. The comparatively poorer performance of the two-component system with nanoparticle (though still significantly higher than that of the nanoparticle-only group) may be attributed to a number of factors, including but not limited to increased nonspecific binding (FIG. 2A-2J), slower crosslinking kinetics (FIGS. 3A-3J), and increased complement activation. While these differences do not significantly influence survival in the liver resection model, they may factor in the selection of trial groups in testing for larger animal models and serve to validate the efficacy of the in vitro assays in screening synthetic hemostats.

To this date, the transfusion of blood products (whole blood, coagulation factors, and blood components) remains the preferred treatment for internal bleeding in the clinic, though this is not always feasible due to the perishability and advanced storage needs of these materials. Researchers have thus sought to address this by engineering synthetic hemostats that mimic blood components, such as peptide-decorated polymers, nanoparticles and liposomes, and have demonstrated the efficacy of these materials across a wide variety of injury models (Lashof-Sullivan, et al., Proc Natl Acad Sci USA 2014, 111, 10293; Hickman, et al., Scientific Reports 2018, 8, 3118). Prior to this study, one approach that has yet to be investigated in nanoparticle-based hemostats is the strengthening of the clot through fibrin-independent mechanisms. Fibrin is critical to the formation of a clot—however, it is also the first of the coagulation factors to fall below critical levels upon blood loss and hemodilution (Morrow, et al., International journal of molecular sciences 2021, 22; Burggraf, et al., World journal of emergency surgery: WJES 2020, 15, 31), ahead of other blood proteins such as thrombin and FVII (Gratz, et al., Journal of clinical medicine 2020, 9). In addition, it is sensitive to effects of the trauma triad beyond coagulopathy: acidosis increases the rate of clot lysis (Dirkmann, et al., The journal of trauma and acute care surgery 2013, 74, 482), while hypothermia further decreases fibrinogen synthesis (Martini, The Journal of trauma 2009, 67, 202). In the absence of stable clot formation—which may occur as part of trauma-induced coagulopathy (Moore, et al., Nature Reviews Disease Primers 2021, 7, 30; Sloos, et al., Intensive Care Medicine Experimental 2022, 10, 1).

There is a significant need to target the site of hemorrhage and strengthen the clot in a fibrin-independent manner

The two-component system proposed herein provides a potential solution to these points. By leveraging increased accumulation of activated-platelet-targeted materials at the wound site—a phenomenon widely reported in literature (Lashof-Sullivan, et al., ACS biomaterials science & engineering 2016, 2, 385; Anselmo, et al., ACS Nano 2014, 8, 11243)—this system applies the concept of the critical gelation concentration to achieve wound-targeted clot strengthening without increased lung accumulation or systemic toxicity. Its targeting mechanism through activated platelets and bioorthogonal crosslinking through azide-DBCO reactions mitigate the effect of fibrin depletion in severe hemorrhage, as demonstrated through enhanced platelet recruitment and overall coagulation potential even in fully hemodiluted environments. This is particularly significant, as the effect of nanoparticle hemostats on clot stability under simultaneously decreased levels of clotting factors, platelets, and fibrinogen has yet to be reported in literature. The decreased therapeutic dose, nonspecific binding, and complement activation of the two-component system relative to the NP-only treatment also suggest that this approach may result in fewer side effects upon translation to more advanced animal models, while its increased survival affirm the effectiveness of this treatment in the context of already highly successful NP-only systems. Future studies may expand towards engaging specific factors of the coagulation cascade or to larger animal models in order to further optimize and gauge the translational potential of this system. Ultimately, the results of this study demonstrate the promise of a multi-pronged approach towards treating internal hemorrhage and introduce the use of enhanced injury accumulation and bioorthogonal crosslinking to establish wound-targeted hemostatic effects.

Summary

In this work, a two-component hemostat for targeted, bioorthogonal crosslinking was developed for the treatment of internal bleeding by functionalization of PEG-b-PLGA nanoparticles with GRGDS and clickable azide moieties. Nanoparticles and multiarm polymers with corresponding copper-free crosslinking groups (DBCO) were delivered as a second component. The two-component system was demonstrated to only result in particle aggregation/significant concentration at several times circulating concentrations, and to enhance both platelet and nanoparticle recruitment at a peptide:azide ratio of 3:1. In addition to that, the system appeared to slow plasmin lysis of fibrin clots as monitored through a one-dimensional capillary assay, recover platelet recruitment and fibrin polymerization in hemodiluted environments, and require a lower therapeutic dose that resulted in decreased complement activation. Biodistribution, pharmacokinetics, and biocompatibility were evaluated in an uninjured model, and no signs of long-term inflammation or skewed biodistribution from the targeted nanoparticles were observed, confirming the safety of the system for systemic delivery. Finally, the two-component system was demonstrated to result in significantly improved survival relative to the nanoparticle-only group in a mouse liver resection model. These findings underscore the importance of supplementing the native hemostasis process in a comprehensive manner, providing guiding principles for the future development of artificial blood replacements.

### Example 4: Demonstration of Safety in Pig Model

### Methods

All experiments were conducted according to protocols approved by the Institutional Animal Care and Use Committee (IACUC) at MGH. Nanoparticles were synthesized at MIT according to previously-described protocols, sterilized with UV, and filtered through a 0.8 micron membrane to remove aggregates. The crosslinker, purchased from CreativePEGWorks, was dialyzed for 24 hours in deionized water and filtered through a 0.2 micron membrane.

Yorkshire swine of 40-55 kg were anesthetized with 1.5% isoflurane, dosed with 100 mg propofol, and intubated. The right carotid artery, right external jugular vein, and left external jugular vein were catheterized, with the Swan-Ganz catheter inserted in the left external jugular vein. A baseline blood sample was taken for the measurement of arterial blood gases (2.5 mL non-heparinized and 0.4 mL heparinized), five minutes before the crosslinker dose was injected (stoichiometric equivalent to nanoparticle doses). Nanoparticles, either functionalized with GRGDS or a GRGDS-cRGD-VBP (vWF-binding peptide) combination, were injected five minutes later. The animals were monitored for a total of 120 minutes (blood drawn at 30-min intervals), before they were euthanized with 11 mL EUTHASOL.

### Results

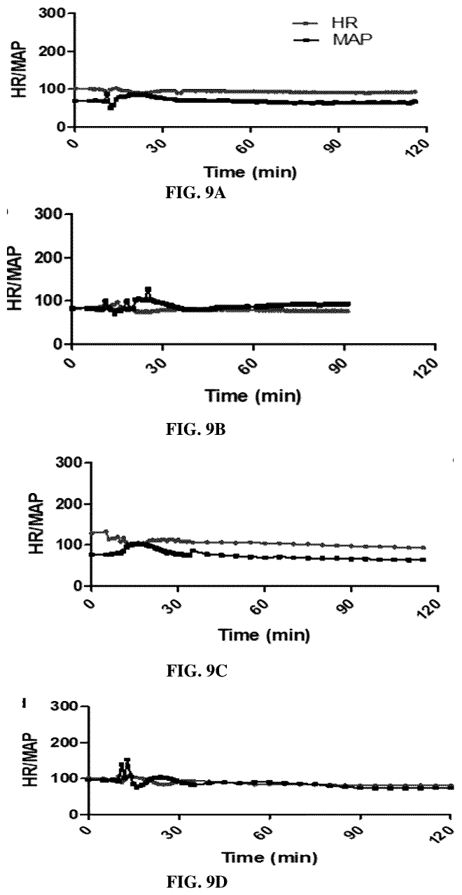

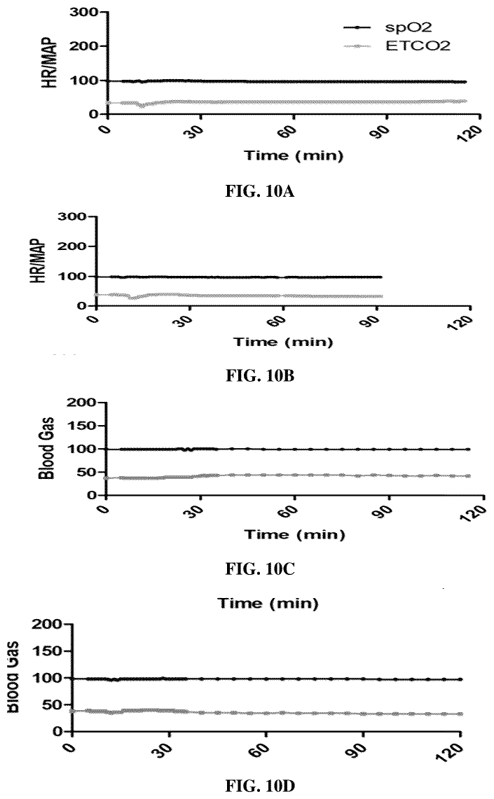

The heart rate, mean arterial pressure, oxygen saturation, and end-tidal carbon dioxide measurements of the four pigs tested are illustrated in FIGS. 9A-9D and 10A-10D.

No long-term changes were observed in heart rate (HR) and mean arterial pressure (MAP), though transient fluctuations were observed in the few minutes following NP injection (t=10 min). No fluctuations were observed following crosslinker injection (t=5 min). Further, no changes in oxygen saturation and end-tidal carbon dioxide was observed following NP injection (t=10 min) or crosslinker injection (t=5 min).

Modifications and variations of these compositions and methods of use thereof will be obvious from the foregoing and are intended to come within the scope of the following claims.

We claim:

1. A composition comprising
an interactive two-component system consisting of a targeting component and a crosslinking component, preferably a dendrimer, a star polymer, a polyelectrolyte or a nanoparticle.

2. The composition of claim 1, wherein the targeting component and the crosslinking component are configured to react with each other.

3. The composition of claim 2, wherein the targeting component and the crosslinking component do not aggregate with each other in solution.

4. The composition of claim 2, wherein the targeting component comprises a polypeptide sequence that comprises a ligand for a receptor on a cell present at a site of injury.

5. The composition of claim 4, wherein the polypeptide sequence binds to a receptor on an activated platelet or a von Willebrand factor.

6. The composition of claim 4, wherein the polypeptide sequence comprises GRGDS (SEQ ID NO:1).

7. The composition of claim 4, wherein the targeting component further comprises a hydrophilic component and a hydrophobic component, wherein the polypeptide sequence is covalently attached to the hydrophilic component.

8. The composition of claim 7, wherein the hydrophilic component is a polyethylene glycol (PEG) molecule.

9. The composition of claim 7, wherein the hydrophobic component is selected from the group consisting of polyesters, polyacrylates, poly(meth)acrylates, and polyurethanes.

10. The composition of claim 7, wherein hydrophobic component comprises a poly(D,L-lactide-co-glycolide) (PLGA).

11. The composition of claim 2, wherein the crosslinking component comprises a corresponding moiety reacting with the targeting component.

12. The composition of claim 11, wherein the corresponding moiety to react with the targeting component comprises a bioorthogonal click-crosslinking group.

13. The composition of claim 12, wherein the bioorthogonal click-crosslinking group comprises an azide.

14. The composition of claim 12, wherein the bioorthogonal click-crosslinking group comprises a dibenzylcyclooctyne (DBCO).

15. The composition of claim 1, wherein (i) the targeting component and the crosslinking component comprise nanoparticles, or (ii) the targeting component comprises nanoparticles and the crosslinking component comprises a small molecule crosslinker, a polymeric crosslinker, preferably a polymeric crosslinker, wherein the small molecule crosslinker or polymeric crosslinker is bivalent or multivalent, preferably the polymeric crosslinker is multivalent.

16. The composition of claim 15, wherein the nanoparticles comprise PLGA polymers.

17. The composition of claim 15, wherein the nanoparticles have an average diameter of between 100 and 500 nm.

18. The composition of claim 1, wherein the targeting component and the crosslinking agent are packaged separately in a kit for administration when needed.

19. A method of treating non-compressible hemorrhage, the method comprising administering a therapeutic comprising the composition of claim 1 to a subject with an internal injury, preferably wherein the targeting component is administered separately from the crosslinking component.

20. The method of claim 19, wherein the subject has internal bleeding or incompressible or inaccessible bleeding.

21. The method of claim 19, wherein the composition localizes at the site of the internal injury.

22. The method of claim 19, wherein the composition lowers the threshold for platelet accumulation.

23. The method of claim 19, wherein the composition increases clot stability.

24. The method of claim 19, wherein the composition increases survivability.
